(19) United States
(12) Patent Application Publication
(10) Pub. No.: US 2026/0201448 A1
(43) Pub. Date: Jul. 16, 2026

(54) PASSIVE SENSOR CAPABLE OF DETECTION OF MATERIALS INCLUDING SPECIFIC DNA OR DNA-LIKE STRANDS

(71) Applicant: DNA PROFILE LTD., Tel-Aviv (IL)

(72) Inventor: Roberto Igal CHERTKOW, Ashdod (IL)

(21) Appl. No.: 19/132,770

(22) PCT Filed: Nov. 7, 2023
(86) PCT No.: PCT/IL2023/051142

§ 371 Date: May 23, 2025

Related U.S. Application Data
(60) Provisional application No. 63/428,087, filed on Nov. 27, 2022.
(60) Provisional application No. 63/435,258, filed on Dec. 25, 2022.

(51) Int. Cl. C12Q 1/6825; G01N 21/64; G01N 27/22

(52) U.S. Cl. CPC C12Q 1/6825; G01N 21/6428; G01N 27/226; G01N 27/227; G01N 2021/6439

(57) ABSTRACT

A system for detecting DNA within a sample is disclosed. The system includes: transmitter-receivers configured to transmit and receive electromagnetic signals; and a sensor device having sensor elements coupled to the transmitter-receivers, such that electromagnetic signals are transmitted by a transmitter-receiver, pass through the sensor elements, and return to either the same or a different transmitter-receiver. Each sensor element comprises single-stranded DNA fragments. Each single-stranded DNA fragment of a sensor element is configured to couple to a specific single-stranded DNA fragment from the sample to form a double-stranded DNA fragment. Coupling of a single-stranded DNA fragment from the sample to a corresponding single-stranded DNA fragment from the sensor element causes a physical change in signals that return to a transmitter-receiver. A system for detecting a material applies similar principles to detect physical changes resulting from matching of the material within the sample to a material within a sensor.

### RELATED APPLICATIONS

This Application claims priority to U.S. Provisional Patent Application No. 63/428,087, filed Nov. 27, 2022, entitled “Passive Sensor Capable of Detection of Materials Including Specific DNA or DNA-Like Strands,” and to U.S. Provisional Patent Application No. 63/435,258, filed Dec. 25, 2022, entitled “Passive Sensor Capable of Detection of Materials Including Specific DNA or DNA-Like Strands,” the contents of which are incorporated by reference as if fully set forth herein.

### FIELD OF THE INVENTION

The present invention relates to the sensing of materials. More particularly, the invention relates to a sensor capable of recognizing different strands or strand fragments of cDNA/DNA, or RNA, which operates passively without requiring an internal source of power.

### BACKGROUND OF THE INVENTION

Various methods are available for the detection of RNA, DNA or DNA-like molecules. Such methods include, gel electrophoresis and binding the DNA to DNA-specific fluorescent dyes. These methods generally require transporting the sample to a laboratory with dedicated equipment and highly qualified personnel. These methods are too inefficient and too expensive to be useful on a mass scale.

As personalized treatments are developed for people with specific DNA sequences or microbiomes, it is becoming increasingly desirable to detect specific DNA or DNA-like sequences. For example, capabilities are being developed to enable matching between the genomic properties of an individual and the microbiome of the individual, and food or cosmetic products that are best adapted for the individual.

There is accordingly a need for a simple, low-cost, disposable, passive sensor, which does not require an internal power source, for detecting cDNA/DNA/RNA like molecules.

### SUMMARY OF THE INVENTION

The present disclosure teaches a sensor capable of conducting tests of a material in general and RNA/DNA/DNA-like molecules in particular, without the involvement of highly trained, expensive personnel using costly laboratory equipment. The sensor is simple, cheap, and disposable. In addition, the sensor does not require an on-sensor power supply, complex electronic microchips, a microprocessor, multiplexers and complex receivers-transmitters. The sensor may be implemented, inter alia, in the fields of microbiome and personalized beauty, to enable matching between the genomic properties of an individual or his microbiome and food or cosmetic products that are best adapted to that individual.

According to a first aspect, a system for detecting DNA within a sample is disclosed. The system includes: a plurality of transmitter-receivers configured to at least one of transmit and receive electromagnetic signals at a plurality of frequencies; and a sensor device having a plurality of sensor elements coupled to the plurality of transmitter-receivers, such that electromagnetic signals are transmitted by a transmitter-receiver, pass through the sensor elements, and return to either the same or a different transmitter-receiver. Each sensor element comprises a plurality of single-stranded DNA fragments, and each single-stranded DNA fragment of a sensor element is configured to couple to a specific single-stranded DNA fragment from the sample to form a double-stranded DNA fragment. Coupling of a single-stranded DNA fragment from the sample to a corresponding single-stranded DNA fragment from the sensor element causes a physical change in a range of signals that return to the same or the different transmitter-receiver. The term “plurality” as used herein includes a single element as well.

In another implementation according to the first aspect, each sensor element comprises a resonance circuit including a capacitor and an inductor, said capacitor comprising a plurality of electrode plates. The single-stranded DNA fragment of the sensor are adhered to the sensor element at a gap between the plates of the capacitor.

Optionally, each of the plurality of transmitter-receivers comprises a power source, a signal processor for converting power from the power source into electromagnetic signals, an oscillator for generating electromagnetic signals of a plurality of different frequencies, and a signal detector for measuring signals received by the transmitter-receiver.

Optionally, each of the plurality of transmitter-receivers further comprises a first inductive element, and the sensor device further comprises a second inductive element connected to the plurality of sensor elements. The first inductive element and second inductive element are inductively coupled to each other. Electromagnetic signals generated by a respective transmitter-receiver pass to the plurality of sensors of the sensing device via the coupled inductive elements. Electromagnetic signals pass from the plurality of sensor elements of the sensor device to the signal detector of a respective transmitter-receiver via the coupled inductive elements.

Optionally, the second inductive element is a fractal inductor.

Optionally, the second inductive element is wired to a coplanar waveguide comprised of signal conductors and ground conductors, wherein each sensor element is coupled to at least one of the signal and ground conductors.

Optionally, when a frequency of a signal transferred to a sensor device by the coplanar waveguide matches a frequency of the resonance circuit of a sensor element, the sensor element enters into resonance, and the resonance circuit acts as a band stop filter or band pass filter, thereby blocking transmission of the signal between the sensor device and the at least one transmitter-receiver, thereby causing a dip in signals of said frequency measured by the signal detector of said transmitter-receiver.

Optionally, for each of the plurality of sensor elements, when the single-stranded DNA fragment from the sample is not coupled to the single-stranded DNA fragment from the sensor and therefore not forming a double-stranded DNA fragment, the band stop filter or band pass filter prevents transmission of a first resonant frequency to the transmitter-receiver, and when the single-stranded DNA fragment from the sample is coupled to the single-stranded DNA fragment from the sensor, the band stop filter or band pass filter prevents transmission of a second resonant frequency that is different from the first resonant frequency to the transmitter-receiver.

Optionally, each of the plurality of sensor elements has a different resonant frequency relative to the others of the plurality of sensor elements when a respective double-stranded DNA fragment is coupled therein. When a transmitter-receiver transmits a range of signals having a plurality of frequencies, including frequencies in which one or more of the sensor elements has a resonant frequency, a signal detector detects each of the frequencies within the range at which a dip in power is measured.

Optionally, the single-stranded DNA of each sensor element is attached to beads which are placed between the plates of the capacitor.

Optionally, the beads are made of a conductive material.

Optionally, the beads are formed of a layer of conductive material that is coated by an isolating layer.

Optionally, a filler material is within the layer of conductive material.

Optionally, the beads are stacked within the gap to thereby create a packed structure of beads.

Optionally, within each gap, a plurality of different types of single-stranded DNA fragments are attached to the beads in different ratios, thereby enabling determination of presence of more than one type of single-stranded DNA fragment from the sample, based on a magnitude of a change in capacitance.

Optionally, the electrode plates are arranged in one or more of the following configurations: parallel plate capacitor; interlacing comb of finger plates; or at least one electrode is a tip with a small apex. Optionally, the capacitor is a parallel-plate capacitor and the single-stranded DNA is adhered between the plates of the parallel-plate capacitor.

In another implementation according to the first aspect, the plurality of transmitter-receivers are configured to transmit and receive optical signals. The sensor device comprises an optical transmission line for transmitting and receiving optical signals. Each sensor element comprises an optical coupler including a gap between two adjacent waveguides. The optical coupler permits transfer of light of a particular coupling frequency across the gap. The single-stranded DNA of each sensor element is adhered within the gap.

Optionally, when a frequency of a signal being transferred through the optical transmission line matches the coupling frequency of the optical coupler of a given sensor element, the signal at that frequency is transferred between the waveguides of said sensor element, thereby preventing further transmission of the signal along the transmission line, causing said frequency to not be received by a respective transmitter-receiver.

Optionally, for each of the plurality of sensors, when the single-stranded DNA fragment from the sample is not coupled to the single-stranded DNA fragment from the sensor, the optical coupler prevents transmission of a first coupling frequency to the transmitter-receiver, and when the single-stranded DNA fragment from the sample is coupled to the single-stranded DNA fragment from the sensor, the optical coupler prevents transmission of a second coupling frequency that is different from the first coupling frequency to the transmitter-receiver.

Optionally, each of the plurality of sensor elements has a different coupling frequency for the optical coupler relative to the others of the plurality of sensor elements when a respective double-stranded DNA fragment is configured in the gap. When the transmitter-receiver transmits light in a plurality of frequencies, including coupling frequencies of each of the optical couplers, the signal detector detects each of the frequencies at which transmission is prevented.

Optionally, advancing of a light signal through the optical transmission line induces a secondary light emission due to phosphorescence or fluorescence induced on the single-stranded or double-stranded DNA of each sensor element. The secondary light emission is detectable by a signal detector of a transmitter-receiver.

Optionally, the plurality of transmitter-receivers are configured to emit and receive light of multiple frequencies simultaneously.

In another implementation according to the first aspect, introduction of material to the sensor elements that is not a single-stranded DNA sample capable of coupling to the single-stranded DNA of the sensor element causes no change in a range of signals that return to the same or different transmitter-receiver, or a different change in the range of signals that return to the same or different transmitter-receiver compared to introduction of the specific single-stranded DNA fragment from the sample.

According to a second aspect, a method for detecting DNA within a sample is disclosed. The method includes: transmitting electromagnetic signals from a plurality of transmitter-receivers to a sensor device containing a plurality of sensor elements, wherein the sensor elements are coupled to the plurality of transmitter-receivers such that electromagnetic signals are transmitted by a transmitter-receiver, pass through the sensor elements, and return to either the same or a different transmitter-receiver; wherein each sensor element comprises a single-stranded DNA fragment, and said single-stranded DNA fragment of the sensor element is configured to couple to a specific single-stranded DNA fragment from the sample to form a double-stranded DNA fragment, wherein, coupling of a single-stranded DNA fragment from the sample to a corresponding single-stranded DNA fragment from the sensor element causes a physical change in a range of signals that return to the same or a different transmitter-receiver; detecting the electromagnetic signals received by the plurality of transmitter-receivers; and based on whether a particular electromagnetic signal that was sent by a transmitter-receiver is subsequently received by the same or a different transmitter-receiver, determining whether a particular single-stranded DNA fragment from the sample is coupled to a corresponding single-stranded DNA fragment from the sensor element.

In another implementation according to the second aspect, each sensor element comprises a resonance circuit including a capacitor and an inductor. The single-stranded DNA of the sensor element is adhered to the sensor element at a gap between the plates of the capacitor. Each transmitter-receiver comprises a power source, a signal processor for converting power from the power source into electromagnetic signals, an oscillator for generating electromagnetic signals of a plurality of different frequencies, and a signal detector for measuring power received by the transmitter-receiver. A first inductive element is in each respective transmitter-receiver and at least one second inductive element is within the sensor device and connected to the plurality of sensors, wherein each first inductive element is inductively coupled to a second inductive element. The method further includes: generating electromagnetic signals in at least one of the transmitter-receivers; passing the electromagnetic signals from the transmitter-receiver to the sensor device via an inductive coupling between the first inductive element of said transmitter-receiver and a second inductive element; and passing the electromagnetic signals, via an inductive coupling, from the plurality of sensor elements to the signal detector of the same or a different transmitter-receiver.

Optionally, when a frequency of a signal transferred to the sensor device by the first and second inductive elements matches a frequency of the resonance circuit of a sensor element, the sensor element enters into resonance, and the resonance circuit acts as a band stop filter or band pass filter, thereby blocking transmission of the signal to the signal detector, causing a dip in power measured by the signal detector.

Optionally, the method further includes, for each of the plurality of sensor elements, when the single-stranded DNA fragment from the sample is not coupled to the single-stranded DNA fragment from the sensor, preventing transmission of a first resonant frequency to the transmitter-receiver, and when the single-stranded DNA fragment from the sample is coupled to the single-stranded DNA fragment from the sensor, preventing transmission of a second resonant frequency that is different from the first resonant frequency to the transmitter-receiver.

Optionally, each of the plurality of sensor elements has a different resonant frequency relative to the others of the plurality of sensor elements when a respective double-stranded DNA fragment is configured between plates of the capacitor, and the method further includes: transmitting, with at least one of the plurality of transmitter-receivers, a range of signals of a plurality of frequencies, including frequencies in which one or more of the sensor elements has a resonant frequency; and detecting, with the signal detector of at least one of the transmitter-receivers, each of the frequencies at which a dip in power is measured.

Optionally, the plurality of transmitter-receivers are configured to transmit and receive optical signals; the sensor device comprises an optical transmission line for transmitting and receiving optical signals; each sensor element comprises an optical coupler including a gap between two adjacent waveguides, wherein the optical coupler permits transfer of light of a particular coupling frequency across the gap, and the single-stranded DNA of each sensor element is adhered within the gap. The method further includes: generating optical signals with at least one of the plurality of transmitter-receivers; transferring the optical signals from the at least one transmitter-receiver to the plurality of sensor elements via the optical transmission line; and transferring the optical signals from the optical transmission line to the signal detector of at the at least one transmitter-receiver or a different transmitter-receiver. When a frequency of a signal being transmitted through the optical transmission line matches the coupling frequency of the optical coupler of a given sensor element, the signal at that frequency is transmitted between the waveguides of said sensor element, thereby preventing further transmission of the signal along the transmission line, causing said frequency to not be received by a respective transmitter-receiver.

Optionally, the method further includes, for each of the plurality of sensor elements, when the single-stranded DNA fragment from the sample is not coupled to the single-stranded DNA fragment from the sensor element, preventing transmission of a first coupling frequency to the signal detector, and when the single-stranded DNA fragment from the sample is coupled to the single-stranded DNA fragment from the sensor, preventing transmission of a second coupling frequency that is different from the first coupling frequency to the signal detector.

Optionally, each of the plurality of sensor elements has a different coupling frequency for the optical coupler relative to the others of the plurality of sensor elements when a respective double-stranded DNA fragment is configured in the gap. The method further includes: transmitting light with the at least one transmitter-receiver in a plurality of frequencies, including coupling frequencies of each of the optical couplers, and detecting, with the signal detector, each of the frequencies at which transmission is prevented.

Optionally, the method further includes: inducing a secondary light emission due to phosphorescence or fluorescence on the single-stranded or double-stranded DNA of each sensor element; and detecting the secondary light emission by a signal detector of a transmitter-receiver.

Optionally, the method further includes emitting and receiving light of multiple frequencies simultaneously.

According to a third aspect, a system for detecting a sample material within an agglomerate of sample materials is disclosed. The system includes: a sensing device comprising a plurality of detecting elements, each detecting element being specific for a particular material to be detected, and one or more external transmitter-receivers. The sensing device is configured to output a detecting signal to the one or more external transmitter-receivers, signifying a positive detection of the material to be detected, when an introduced corresponding material matches the first material in at least one of geometrical configuration and chemical properties. The sensing device is configured to not output the detecting signal signifying a positive detection to the one or more external transmitter-receivers when the introduced corresponding material does not match the first material in at least one of geometrical configuration and chemical properties.

In another implementation according to the third aspect, each detecting element comprises a capacitive element including actual or theoretical electrodes or plates; and a first material between the electrodes or plates. The presence and amount of the first material measurably affects a capacitance of the capacitive element. Introduction of a corresponding material to be detected between the electrodes or plates, and thereby generating of a second material comprising the first material and the material to be detected, measurably affects the capacitance of the capacitive element relative to the capacitance of the detecting element in a presence of only the first material, causing a difference in measured parameters of capacitance.

Optionally, the first material is a light or radiation sensitive substance that is sensitive to a particular range of radiation, and the material to be tested is radiation. Introduction of radiation of a particular spectrum changes properties of the first material, which thereby affects the measured parameters of capacitance.

Optionally, the first material is a chemical substance that is sensitive to reaction with a specific chemical substance to be tested, and the material to be tested is a chemical substance. Introduction of the chemical substance to be tested induces a chemical reaction in the first material, which thereby affects the measured parameters of capacitance.

Optionally, the first material is a hygroscopic substance that is reactive to presence of water drops or vapor, and the material to be tested is water drops or vapor. Introduction of the water drops or vapor induces a chemical reaction in the first material, which thereby affects the measured parameters of capacitance.

Optionally, the sensing device further comprises a channel between the electrodes or plate, the first material is positioned at a specific location within the channel, the channel comprises an inlet and an outlet, and a means for advancing materials from the inlet to the outlet. When corresponding material to be tested is drawn from the inlet to the outlet, a change in capacitance is induced when the material to be tested reaches and interacts with the first material.

Optionally, the first material is configured within a gel substance, the material to be tested is a DNA-like fragment, and the means for advancing materials is electrophoresis.

### BRIEF DESCRIPTION OF THE DRAWINGS

FIGS. 1A-1C illustrate the general concept underlying a detector, according to embodiments of the present disclosure;

FIGS. 2A-2C illustrate the general concept of a multi-sensing device for sensing multiple samples, according to embodiments of the present disclosure;

FIGS. 3A-3C illustrate architecture of a multi-sensing device, according to embodiments of the present disclosure;

FIG. 4 illustrates sensor internal architecture and data flow, including multiplexing and demultiplexing of data from the multi-sensing device of FIGS. 3A-3C, according to embodiments of the present disclosure;

FIGS. 5A-5C illustrate a detection device for DNA-like material, according to embodiments of the present disclosure;

FIGS. 6A-6B illustrate a DNA sensor device concept, according to embodiments of the present disclosure;

FIGS. 7A-7C illustrate a capacitor as a sensor component, according to embodiments of the present disclosure;

FIGS. 8A-8C illustrate LC resonant circuit measurement and detection, according to embodiments of the present disclosure,

FIGS. 9A and 9B illustrate different embodiments of transmitters and receivers, according to embodiments of the present disclosure,

FIGS. 10A and 10B illustrate a planar strip embodiment of the sensor device configured for proximity coupling, according to embodiments of the present disclosure,

FIGS. 11A-11D illustrate the proximity coupling planar strip of FIGS. 10A-10B with multiple sensing sites, according to embodiments of the present disclosure,

FIGS. 12A-12D illustrate an optical domain planar sensor, according to embodiments of the present disclosure,

FIGS. 13A-B illustrate a concept for detection of DNA-like fragments, according to embodiments of the present disclosure;

FIGS. 14A-C illustrate increase of dielectric presence, according to embodiments of the present disclosure;

FIGS. 15A-15C illustrates use of microbeads to enhance detection of biological species and DNA-like molecules, according to embodiments of the present disclosure,

FIGS. 16A-I illustrate enhancement of detection using metallic beads, according to embodiments of the present disclosure; and

FIGS. 17A-C illustrate geometrical configurations of capacitor electrodes, according to embodiments of the present disclosure;

FIGS. 18A-B illustrate the use of fractal inductors as part of a planar sensing device, according to embodiments of the present disclosure.

FIGS. 19A-19D illustrate the mechanical structure of conceptual materials that are detected, according to embodiments of the present disclosure;

FIGS. 20A-20D illustrate additional examples of conceptual materials that are detected, according to embodiments of the present disclosure;

FIGS. 21A-21B illustrate substrate-less continuous monitoring, according to embodiments of the present disclosure;

FIGS. 22A-C illustrate sensing devices with a plurality of transmitter-receivers separated, including capacitive coupling instead of or in addition to inductive coupling, according to embodiments of the present disclosure; and

FIGS. 23A-B illustrate sensing devices with different forms of coupling between the sensing elements and the transmitter and receiver elements exemplifying a contacting coupling, according to embodiments of the present disclosure.

### DETAILED DESCRIPTION OF THE INVENTION

The present invention relates to sensing of materials. More particularly, the invention relates to a sensor capable of recognizing different strands or strand fragments of cDNA/DNA, or RNA, which operates passively without requiring an internal source of power.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. In particular, throughout this disclosure, when the disclosure described that an element “may” be present, it is understood that the described element is not necessarily present, and the element may be replaced with an equivalent element or not be present at all. Likewise, when a list of specific examples is given, the list is not necessarily exclusive, even without a disclaimer such as “including but not limited to,” and other suitable examples may be utilized.

As used in the present disclosure, the term “sensor” refers to an element that immediately responds to the presence or absence of an analyte material. The term “sensor device” refers to a device that contains the sensor, and which further includes components that enable transmission of the status of the sensor to a user.

The sensor device described herein, in certain embodiments, is configured for sensing of DNA samples. In advantageous embodiments, the DNA samples are collected from a layman subject, for example, by spitting into a tube. The DNA samples are then isolated and brought to the sensor for testing. Exemplary apparatuses and methods for isolating and bringing the DNA samples to the sensor are disclosed at length in the U.S. Provisional Applications to which the present application claims priority. For the purposes of the present disclosure, it is assumed that the DNA sample is already isolated and ready for sensing.

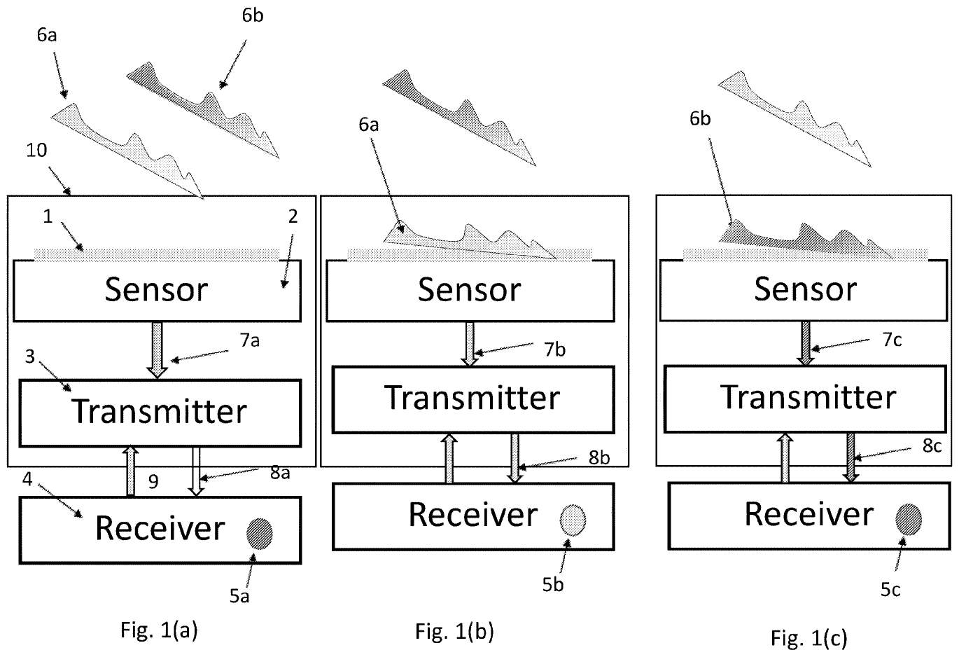

Referring to FIGS. 1A-1C, the basic concept of a sensor device 10 is explained. Sensor device 10 contains a sensing component 1 and a transmitter 3. Sensing component 1 is configured to detect some characteristic of sample 6a. As shown in FIG. 1A, when samples 6a, 6b are not in detecting contact with sensing component 1, the sensor 2 is in a non-detecting state. Thus, a non-detecting signal 7a is sent to the transmitter 3 of the device 10. Alternatively, no signal is sent to the transmitter 3, with the absence of a signal being indicative of non-detection. An external receiver 4 monitors the transmitter 3 of the device and occasionally may send control or other signals 9 to the device through the transmitter 3. When there is no detection by the device 10, a signal 8a may be transmitted to the receiver 4. In such a case, the receiver 4 may set an indicating component to a non-detecting status 5a which may serve as a baseline.

In FIG. 1B, sample 6a enters into sensing proximity with sensing component 1, which is intended and made specifically to detect some characteristic of sample 6a. The sensor device turns into a “detected” mode and a signal 7b communicates the status to transmitter 3. Transmitter 3, either by itself (i.e., self-triggered or self-initiated) or as a consequence of an interrogation signal 9 from the receiver, sends a confirmation signal 8b to the receiver, indicating a positive detection. The receiver in turn, may set the indicating component into a detecting status 5b. Although in this and the subsequent Figures, the indicating component is presented as a schematic shape having a particular color, the indication may take any form, including but not limited to a textual indication.

In the case that a different sample 6b that is not intended to be sensed by sensing component 1 comes into detecting contact with detecting component 1, one of two scenarios may apply. In a first scenario, no detection occurs at all, and as a consequence the case of FIG. 1A applies and a non-detection status is set by the receiver. Alternatively, as indicated in FIG. 1C, a different signal 7c is sent by the sensor 2 to the transmitter 3. Signal 7c represents a faulty or wrong detection state. In turn, the transmitter 3, either as a consequence of a control signal 9 sent by the receiver 4 or as a response of its own algorithm that may have been triggered by signal 7c, sends a signal 8c to receiver 4. The receiver 4 in turn may indicate the faulty or non-intended detection state 5c.

The sensor device of FIGS. 1A-1C is thus directed to detect a specific type of sample and discard or disregard other possible samples. It is desirable, however, that a sensor device be capable of responding to various samples being presented together to the device. In such a case, an upgraded concept is required as follows.

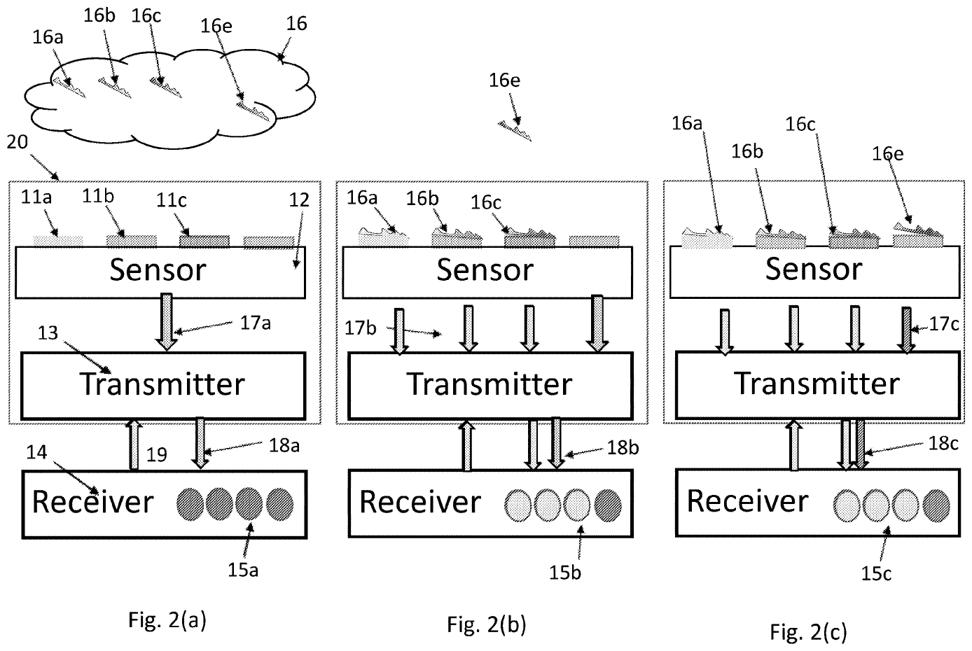

Referring to FIGS. 2A-2C, the basic concept of a device 20 capable of sensing one or more samples in parallel is explained. Device 20 contains several sensing components, 11a, 11b etc., similar to sensing component 1 of FIG. 1. Each sensing component 11a, 11b, etc. is capable of detecting only one kind of sample 16a, 16b, etc. In the test agglomerate 16 to be presented to the test device 20, there may be none, one or several of the samples 16a, 16b, 16c, etc. The test agglomerate 16 might also contain other samples 16e that are not detectable by the specific variant of device 20, but might be detectable by other variants of device 20. As used herein, “not detectable” means that among the sensing components 11a, 11b, 11c, etc. there is no detecting component 11e for sample 16e.

As explained for the device 10 of FIGS. 1A-1C, if the samples or test agglomerates are not in detecting contact with a detecting component, the sensor 12 is in a non-detecting state. A non-detecting signal 17a may optionally be transferred to the transmitter 13 of the device 20. An external receiver 14 monitors the transmitter 13 of the device and may occasionally send control or other signals 19 to the device through the transmitter 13. In the case that there is no detection by the device 20, a signal 18a may be transmitted to the receiver 14. In such a case, the receiver 14 may set an indicating component to a non-detecting status 15a. Because device 20 is capable of detecting multiple samples, the indicating component may include multiple sub-components that are configured to indicate the status of detection of each of those samples, or may be a single component that indicates the status of multiple sensors.

In FIG. 2B, samples 16a, 16b, 16c etc. come into a detecting contact with a sensing component 11a, 11b, 11c etc. respectively, which are configured to detect some characteristics of samples 16a, 16b, 16c etc. As a result, the respective sensor components 16a, 16b, 16c turn into “detected” mode. A single signal or many signals 17b inform the status of the sensing component or components to transmitter 13. Transmitter 13, either by itself (i.e., self-triggered or self-initiated) or as consequence of an interrogation signal 19 from the receiver 14, sends one or more confirmation signals 18b to the receiver 14, indicating a positive detection of the sensor 12. The receiver in turn may set none, some, or all the indicating components into a detecting status 15b.

In the case that a different sample 16e, that is not configured to be sensed by any of the sensing components 11a, 11b, 11c etc. of device 20, comes into detecting contact with a detecting component, either of two scenarios may apply. Either no detection occurs at all, and, as a consequence, the scenario of FIG. 2a applies and a non-detection status is set by receiver 14. Alternatively, as indicated in FIG. 2c, a different signal 17c is sent by the sensor 12 to the transmitter 13. In turn, the transmitter sends a signal 18c to receiver 14. The receiver 14 in turn may indicate the faulty or non-intended detection state 15c.

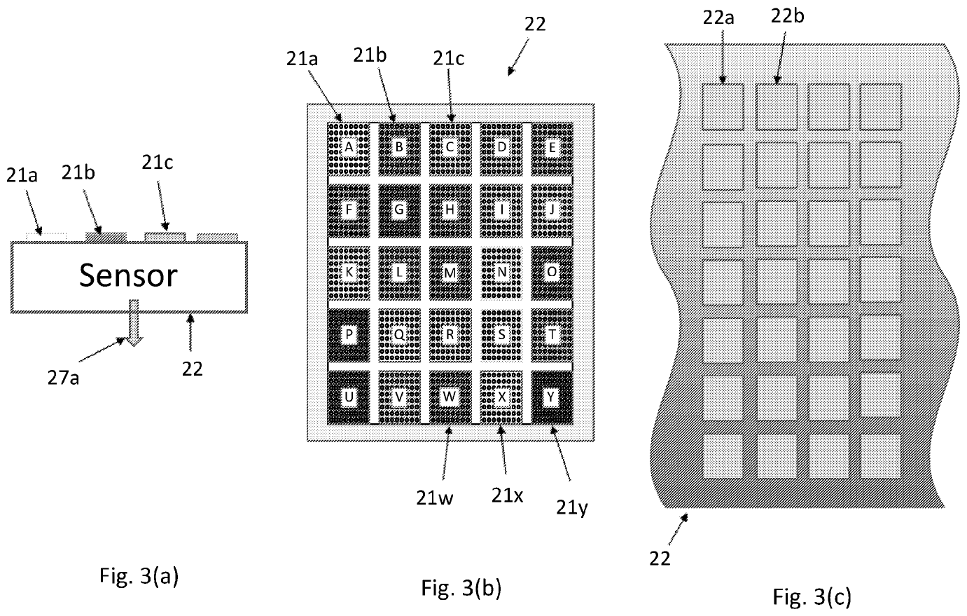

Referring to FIGS. 3A-3C, sensor 22, as shown in FIG. 3A, may have one or many sensing components 21a, 21b, 21c etc. each capable of detecting a specific sample that might be present in the test agglomerate. Signal 27a is configured to serve as a combined indication of the detection status of each of the sensing components. Each of the individual sensing components may be identified in various forms, such as a number, a location within a coordinate system, a location in a running numbers system, a frequency, or a color.

The sensing components are distributed on the sensor in any suitable configuration. In the illustrated embodiments, and without limitation, the sensing components are distributed around the sensor as an array of components. This array can be as a 2D or 3D geometry. Specifically, the sensing components 21a, 21b, 21c . . . 21w, 21x, 21y etc. as shown in FIG. 3b are distributed around the sensor 22 in a 2D array configuration where each such sensing component may detect various distinct samples as indicated by letters A, B, C, . . . etc. These sensing components may detect the same or different samples. For example, sensing element 21a is configured to sense sample A, and sensing component 21x is configured to detect sample X. A and X may be different sample types or the same sample.

Referring to FIG. 3C, the sensors 22 described in FIG. 3B may be sub-components of a larger sensor structure as shown schematically in FIG. 3C. In this context, “schematically” means that the specific dimensions, shapes, and distances between the sensors 22 and sub-structures thereof may vary and need not be exactly as depicted. Each of the sub-structures 22a, 22b, 22c etc. may have the same properties as described previously for sensor 22. In addition, each of the sensor sets 22 may have some common purpose or goal. For example, if sensor set 22a is intended to detect the presence of one bacteria, the sensors 21 within it will recognize fragments available for such bacteria (for example: E coli). Another sensor set such as 22c, may have sensors 21 that are made to detect a different bacteria (for example: Salmonella).

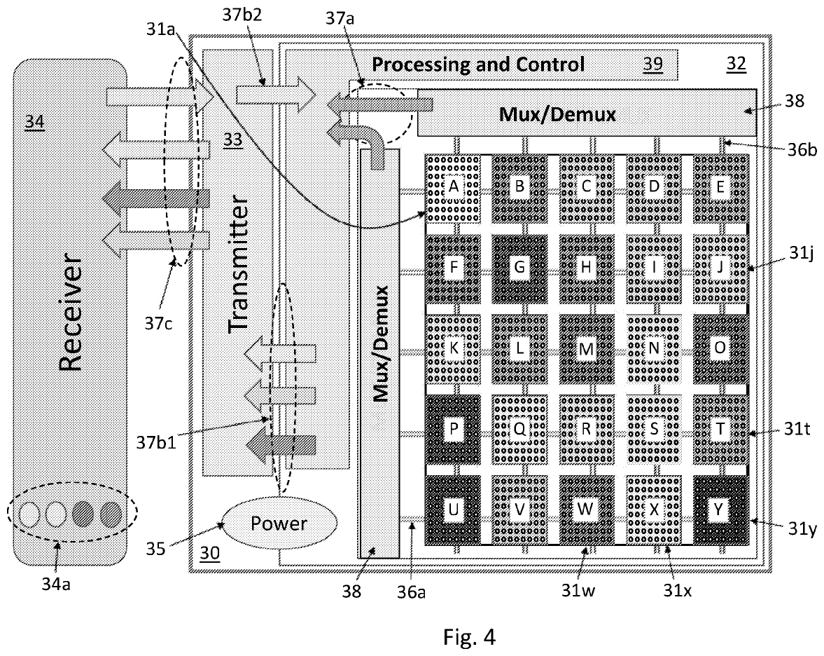

FIG. 4 illustrates a more intricate detail of the sensor structure. Device 30 has a sensor 32 generally having the capabilities and components already described. The sensor has one or more sensing components 31a, . . . 31y, etc. As discussed previously, the sensing components are, for illustrative purposes, arranged in an array. The array may be a physical array, as illustrated schematically, or a logical array. Each sensor component is capable of detecting a specific sample A . . . Y. Information regarding the status of sensing of each component, such as the identification of the sensing component and its state, may be relayed to transmitter 33 through signaling lines.

It is understood that when signals are sent through the signaling lines in parallel (at the same moment), a method is required to separate the signals. To this purpose a multiplexing/demultiplexing (MUX/DEMUX) component 38 is used.

Different forms of a multiplexing/demultiplexing element that are specifically configured for use with DNA or DNA-like samples in general and “materials” as defined throughout this disclosure in general will be discussed further herein. The following paragraphs address general principles relevant to MUX/DEMUX components which may also be implemented with the sensors of the present disclosure.

As is well known to those skilled in the art, MUX/DEMUX is generally implemented by a microelectronic circuit, a specifically purposed microchip, or an embedded circuit. This element, in general, identifies the location in the array of each signal origin and also the information carried by the signal. In this form, it is possible to have the information and state of all of the sensing components of the array in the sensor. For example, the signal lines might be metal conducting lines that are arranged in a row/column setup such that an electric signal will propagate through both of them. Propagation through both of the signal lines is a known general implementation of a matrix addressing schema (see the arrangement of horizontal lines 36a and vertical lines 36b in FIG. 4) while for a series connection the nodes are sequentially arranged along the lines. Conversely, the signal lines might be some kind of waveguides that transfer one or more signals to the multiplexers in an actual or simulated array. “Simulated array,” in this context, means that the physical placement of the elements might not look as an array but their underneath wiring resembles an array in terms of the addressing and signal propagation like the wiring as drawn in the figure. Similarly, the array may be simulated in respect to a row/column arrangement, meaning that the physical arrangement or the physical connections might not be actually a row-column arrangement, but logically they may be addressed or seem to be addressed as components of a vector or array. Furthermore, these waveguides may propagate any range of the EM frequency spectrum, including but not limited to, the light spectrum, X-rays, radio waves, etc. As such, a light signal will propagate through the signal lines to a MUX/DEMUX capable of dealing with optical signals. Even acoustic signals can be made to propagate through respective lines. Furthermore, although the components may be regarded for clarity to be arranged in an array form, the signal lines might not necessary be arranged as rows and columns. Rather, the signal line may be made to be laid out as a single stripe or double or more stripe through all the sensing components of the array in the sensor, in such a way that eventually a MUX/DEMUX element will be able to extract the identification and state of each sensing component of the array. This signal line, as explained before, may be made as a wired electrical connection, a waveguide for EM spectrum including but not limited to the optical spectrum, acoustic waveguide etc.

This being said, MUX/DEMUX element 38 may take the form of an conventional electronic component or a conventional optical component, but it may take any other form that eventually will be able to take the signals received from the sensor component array and identify the sensor component source and provide the identification information to other components of the device such as the control unit, the transmitter or any other component of the device necessary for actually performing as a MUX/DEMUX.

Once the multiplexing/demultiplexing element 38 identifies the signals of the various sensing components, it transfers the information 37a to an optional processing and control unit 39. This unit may, among other functions, control the transfer of the signals from and to the sensor, through the MUX/DEMUX element, and make all the required preparations as required for the functioning of the device. The control unit may include a memory stored on a non-transitory computer-readable medium, and a processor that is configured to execute the instructions stored on the memory to perform the functions described herein.

Control unit 39 transfers signals 37b to/from a transmitter 33. The signals 37b1 being advanced to the transmitter may indicate the status of the sensing components, whether they have nothing detected, had detected a sample, or, if so desired, if a wrong sample reached the wrong sensor component. The signals 37b1 may be sent by the controller, if present, or through the MUX/DEMUX or the sensor components to the transmitter by initiation of any of the elements of the sensor due to a detection event or any other trigger event designated to do so. A signal may also be sent as a consequence of an interrogation or control signal 37b2 sent by the transmitter to the sensor components through any, some, none or all of the control unit 39, MUX/DEMUX 38, signal lines 36 or directly to a sensing component 31.

The signals 37b received from or sent by the sensor 32, by/to transmitter 33; are relayed out of device 30 by way of an appropriate communication path 37c, which may use the EM spectrum, acoustic or other communication means (e.g., standard methods such as NFC, Bluetooth or Wi-Fi or other nonstandard methods such magnetic or electric coupling or acoustic propagation and more methods that this place is short to summarize and some of them are yet to be invented or disclosed), to the receiver 34. In turn, receiver 34 may implement a single or plurality of indicating components 34a. The indicating components 34a may indicate non-detecting status, a detecting status or erroneous detecting status. The indicating components 34a may indicate the status of particular sensing components individually, or may display a combined indication representing the status of multiple sensing components. As discussed, the indicating components may include textual indicators, or symbols, or figures.

The schematic rendering of FIG. 4 further includes a power source 35. This power source 35 might take various forms. Some power sources may be placed in the device, including but not limited to a battery, or a connection to an external power source. Alternatively, the power source might not be in the device itself and the power may be externally relayed and may be provided wirelessly by any possible method such as EM, optical, acoustical by motion or vibration, etc.

In sum, in FIGS. 1A-4, the general workings and structure of a device were disclosed. The device in general is able to detect a sample to be distinguished when the sample is in detecting contact with a sensing component specifically set to detect this sample. When such a detection occurs, the sensor propagates a signal through certain internal elements and subsequently through the transmitter element to an external receiver, which in turn may provide detection/identification information about the sample. If the sample is not in detecting contact with the sensing component, or an unintended sample arrives to a sensor component even if it is in detecting contact with it, no signal or an error reporting signal may be sent to the receiver. As indicated as well, devices may have many sensing components to detect simultaneously many samples present in a test agglomerate. In order to identify and transfer the signals from the sensing components, some kind of MUX/DEMUX element is used. The information identified by the MUX/DEMUX element is transferred to the transmitter and from it to the receiver to be used as needed. All these transitions may be controlled by a processing/controlling element. It is also understood that power is required for all those functions.

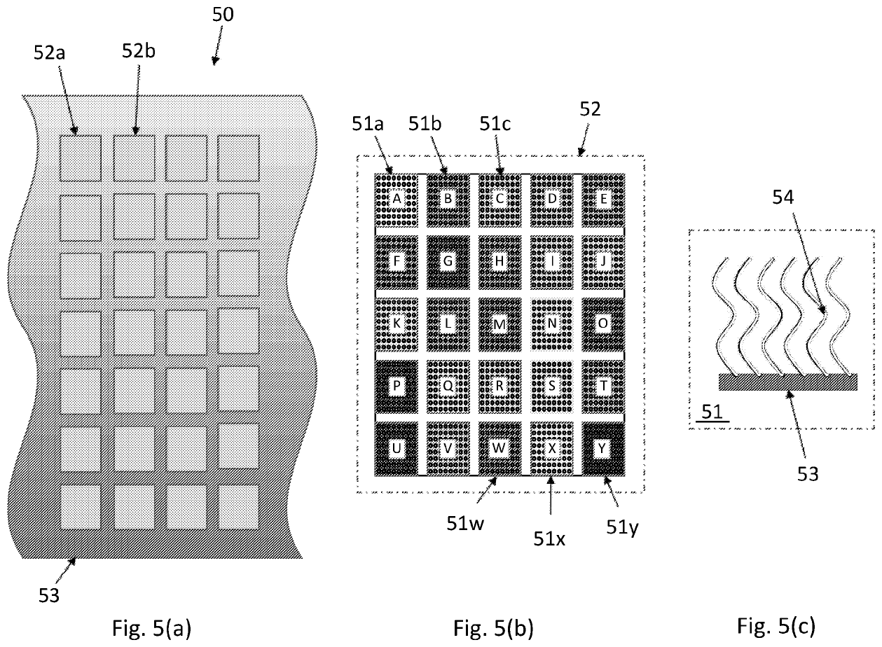

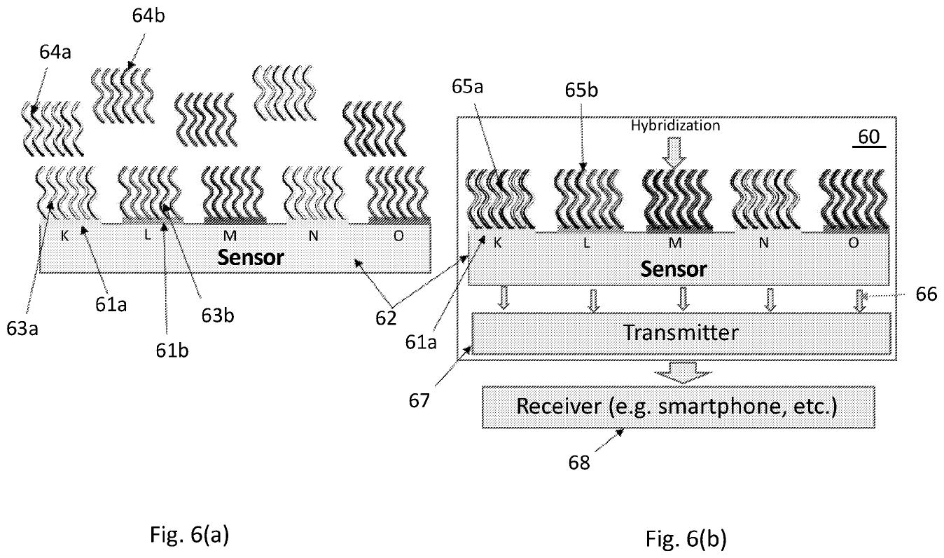

FIGS. 5A-6B introduce implementation of the teachings of this disclosure in regards to the use of the device for identification of various types of generalized “materials” and more specifically to biological or genetic material. As used herein, where DNA fragments are mentioned, RNA fragments can be used as well, either ss (single strand) or ds (double strand) molecules, and in general any type of molecules either organic or inorganic, simple molecules or complex or even polymers etc.

As discussed, an embodiment deals with a device that is capable of identifying DNA-like fragments; for example: from a human microbiome, from a saliva test, for virus detection, etc. As the sample collected may contain many types of DNA or RNA molecules, representing different species to be detected; such as different microbes or various viruses etc.; the device may be able to detect one or more or many such DNA-like samples. The term “sample” refers to a DNA/RNA fragment of any length as required.

Referring to FIG. 5A, device 50 is composed of a set of one or more testing locations 52a, 52b, etc. that can be arranged in any physical configuration and in particular as an array, placed on some holding element 53, which may be any suitable material. Each location 52, being a sensing component, may have one or more sensing elements 51a, 51b, etc. marked A, B, etc., as shown in FIG. 5B. The device 50, and/or each location 52 in it, and/or each sensing element 51 included in a location 52 thereof, may use any of the teachings that were disclosed through this disclosure for any of the components required for its functioning in general and in particular the means of sensing, multiplexing, transmitting the to-be-tested material status. As the intention is to detect DNA like material, the material is RNA/DNA-like molecules. More specifically, each sensing location 51 has some sort of substrate 53, which can be solid, gel or any other type of material, having any form or configuration such as a flat surface, an aggregate of particles such as spheres or cubes; on which DNA fragments 54, cDNA, or oligos, are attached, as shown in FIG. 5C.

The sensing component 52 of FIGS. 5A-C is further detailed in FIGS. 6A-C. Sensor 62 is composed of several sensing elements 61a, 61b etc. marked as K, L, M, etc., on which single strand (ss) oligos or cDNA fragments 63a, 63b etc. are attached. Each such attached fragment is intended to be able to be used to detect a specific DNA-like fragment 64a, 64b etc. that may or may not be present at any sample agglomerate presented to the sensor, as shown in FIG. 6A. In the case that an appropriate ss fragment 64a, 64b, etc. exists in the sample agglomerate and is brought to the vicinity of the sensor 62, and within it to the respective sensing element, for example, by means of chemical procedures, the ss fragment 64a will bond/hybridize to ss cDNA 63a and become a double-stranded (ds) molecule 65a at sensing element 61a designated as K. Similarly, ss fragment 64b will bond/hybridize to ss cDNA 63b and become molecule 65b at sensing element 61b designated as L; and so forth for all the molecules to be tested and sensing elements within sensing device 60 as shown in FIG. 6B. Molecule 65 is thus a new “material” and 63 is a “material” in the broader sense prior to the sensing interaction. In the case that hybridization occurs in any of the sensing elements 61 of sensor 62, as a consequence of this bond/hybridization; the sensor sends a specific signal 66 to transmitter 67 within device 60. Transmitter 67 in turn might communicate with receiver 68, in general and specifically send the detection signals from the sensor. The transmitter and receiver may exchange control information, power supply and detection data, as may be preferable for the implementation. The transmitter with the device 60 may be of any kind; but in particular it may be some kind of RFID device, while the external receiver/transmitter in this case may be a specific dedicated gadget or even a smartphone.

Returning to general principles, the following section will disclose details of how to achieve the various elements described so far. The teachings of the present disclosure are directed to enable a device that can be as simple and therefore as low cost as possible, while still providing the capabilities indicated. To achieve this goal, it will be disclosed how to provide a device with the mentioned capabilities but relying on as few electronic components as possible; as such components are relatively expensive. For this goal, the intension is to avoid an on-device power source, as it is a costly element, and to use a transmitter/receiver that is as simple as possible.

The sensing component was generally described above. As many different kinds of samples are possible with the intended device, a device that can identify various types of samples regardless of their specifics is required. To do so, it is useful to find a common denominator of all the possible samples to be tested. As stated, samples may be: mechanical structures, biological substances, chemical organic or inorganic substances, and more. The common denominator of all these different types of samples is that all the samples may be seen as just some material. Regardless of the specifics of each of the samples, fundamentally they are materials. And as such, the materials each have general properties. These properties include: mechanical properties such as tensile strength, physical properties such as thermal conduction, optical properties such as transparency, sound speed, heat capacity, chemical properties such as oxidation sensitivity, and electrical properties such as conductivity, dielectric and magnetic constants etc. In a preferred embodiment, it would be beneficial for an EM signal to be transferred from the device to a receiver, because electrical physical properties are more straightforward to implement for the common denominator materials. Thus, optical, acoustic, electrical, and magnetic materials properties are the main candidates to be exploited, but others can be considered by those skilled in the art as well.

In preferred embodiments, electromagnetic properties are used: dielectric constant, magnetic permittivity, conductivity, charge, etc. While looking at those parameters all of them affect in some way electrical circuits in the broad sense of the word, meaning: simple conducting circuits for low frequencies, waveguides of various kinds and types in all the frequency ranges up to the optical, X-ray, etc. Therefore, the conceptual idea behind disclosed embodiments is to use the material property in a way that it can identify the sample to be tested among other samples. Stated differently, if any sample of the mentioned samples is regarded as having electrical physical properties as explained, then measuring of the property should identify the sample.

The measurement of the material parameters in the EM spectrum can end up in different practical formats. For the ideas shown in this disclosure, eventually, the sensing is performed through the measurement of characteristic frequencies of the sensors. These characteristic frequencies are a consequence of resonating frequencies of resonators adapted for the specific EM frequency range. These resonators may act as band stop filters or band pass filters. The following section illustrates embodiments based on different parts of the electromagnetic spectrum: a first embodiment based on resonance circuits in the radiofrequency range, and a second embodiment based on optical couplers for the optical range. In the first case, for a RF circuit range, the filter is a LC resonator which prevents the passage of a specific frequency passing through a waveguide. In the second case, a light coupler prevents the transfer of the specific light frequency by forming a “resonant” gap specific to the frequency whose passage it is desired to prevent. These embodiments are applicable for sensing of all types of “materials,” but special emphasis will be given for sensing of DNA or DNA-like samples, as discussed.

Throughout the discussion of these embodiments, conceptually, the resonator or coupler can be mutually interchanged, as the coupler can be seen as a resonator and a resonator drawing energy from a waveguide can be seen as a coupler. The actual naming and references to each other is relative to the range of the EM spectrum being used and for each a naming convention has been adapted accordingly.

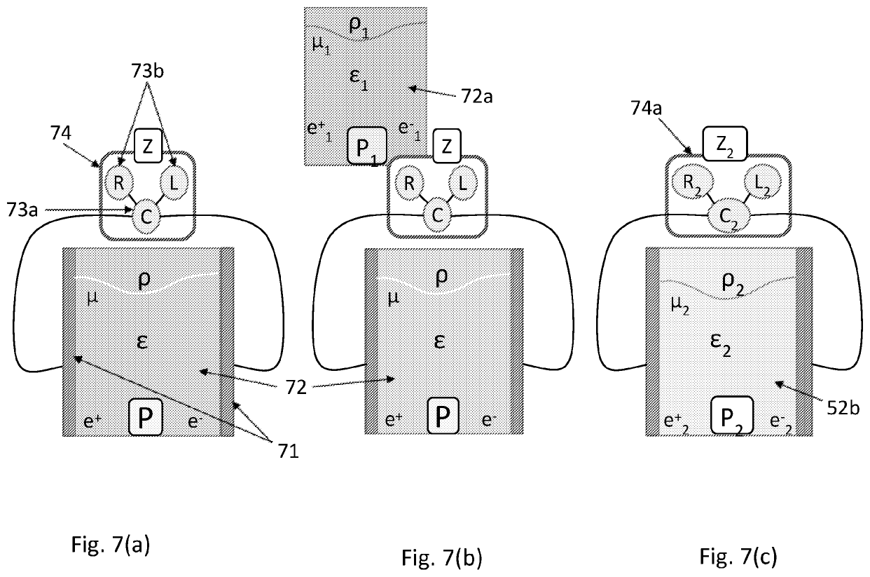

Referring to FIGS. 7A-7C, a capacitor is generally composed of two conducting plates 71, between which a vacuum or a material 72 is placed. The material properties, P, affect the capacitance of the capacitor. The relevant property of the capacitor is the dielectric constant (E) of material 72. A sensor component based on a capacitor may probe the changes in dielectric constant between the plates of the capacitor and identify the sample accordingly. However, as not all the materials have ideal properties, an actual capacitor performance will be affected by various properties of the material beyond the dielectric constant (ε), such as the resistance (ρ) that may be caused, for example, by leaks (visualized as a curved line between plates 71), magnetic permeability (μ) especially while RF signals are involved, and even charges (e−/e+) on the material that will have an impact on the capacitor's overall performance. Regardless, the total behavior of a capacitor is able to identify a sample placed between the capacitor plates.

The performance of the capacitor may be measured by measurement tool 74, which may measure the capacitance (C) 73a, as well as other parameters 73b such as inductance (L) and resistance (R). The measured value provided by the measurement tool 74 is designated as Z. Among the measuring tools are capacitance meters, multimeters etc., and also other methods such as optical tools etc. In one specific example, the measured value Z can be seen as the impedance of the capacitor, affected by the entire set of parameters affecting it. In the case that Z is regarded as an impedance, impedance measuring methods may be used to evaluate the capacitance characteristics. The wire lines in the figure are only illustrative and may symbolize an actual electrical connection but they may symbolize any other interaction between the capacitor and the measuring tool. It is also understood that the plates 71 of the capacitor need not be actual plates, made of conductive or semiconductive or other material; they may be actually present or may be imaginary and exist theoretically as it is implemented in many cases when dealing with electrical components where capacitors and capacitance effect do not need a real actual plate to define a capacitor. Also, the form and configuration of a capacitor need not be as a parallel plate and it may be in any configuration possible for a capacitor like element.

Assuming that a capacitor is provided with a material (which may be a real material, a vacuum or any suitable structure) with parameters P and while measured it provides a value Z, it is defined as the sensing component. With respect to FIG. 7B, a second material 72a is provided, which has material parameters P1 (ε1, ρ1, μ1, e1−/e1+) which represents the sample to be tested.

To test the sample, it is placed within the capacitor plates, either with or without removing the previous material. When placing the sample material to be tested in the sensing component as to be in contact with it, the combined effect of the material with properties P1 and the material with properties P (if it remains in the sensing location), will set the combined material 72b to a new set of properties P2 as in FIG. 7C. The measurement tool 54a will provide values such as capacitance C2, inductance L2, resistance R2 and so forth designated as measured values Z2. The value Z2 will generally be different from value Z, therefore the different value will indicate a positive sensing of sample to be tested 72a by the sensing component. In the case that somehow a different sample, not being the intended sample to be tested, enters the testing region of the measurement it will provide a value that may be different from the value expected while testing a valid sample, therefore indicating a faulty or non-detecting state.

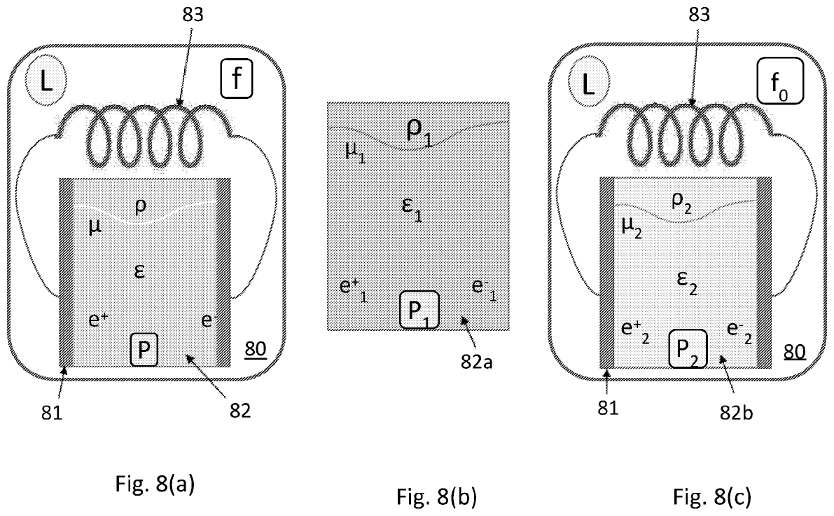

While it is possible to use a measurement tool 74a that is specifically configured to measure capacitance, it is also possible to determine the change in capacitance in other manners. As the proposed embodiment is a non-ideal capacitor, the capacitor will have ideal capacitance but as mentioned, also ideal inductance, resistance, charge etc. Such electrical parameters may be tested by many ways. Referring to FIG. 8A, as an example, an inductor 83 is attached to capacitor 81, so that they form a resonant circuit 80. Resonant circuit 80 is composed of a capacitor 81, as described previously, having material properties (P) 82 and an inductor 83 with inductance L. This circuit, if forced, will start to oscillate between its current and voltage stages, transferring energy from the magnetic to electric states. When the frequency of oscillations attains a certain value, or if the resonator is left to oscillate without being forced, the oscillation settles into a natural frequency, which is the resonant frequency (f) of the resonator. This frequency is characteristic of the components in general and specifically of the capacitance and inductance and resistance of the elements of the circuit. Knowing the inductance L of inductor 83 and the capacitance of capacitor 81, which depends on the properties P of material 82, it is possible to know the value of the frequency of oscillation (f).

In FIG. 8B, there is a sample to be tested 82a, with material parameters P1. The material may be placed in the sensing component (shown in FIG. 8C) at a sensing contact, in the capacitance zone, and the combined material 82b will exhibit properties P2. As the material properties have changed, as explained previously, the capacitance of capacitor 81 will change, changing the overall frequency of the resonator LC to a new value f₀. So, the different frequency between prior to application of the sample to post application of the sample signifies that the sample has been placed in the sensing component, i.e. the capacitor.

Obviously, the capacitor, the inductor and generally the resonant circuit may have various configurations and geometries, which are not illustrated in the Figures. In addition, the resonators may have many realizations, including but not limited to coaxial capacitors and spiral or helix inductors, with micron size dimensions or as planar microstrips, providing the capacitance and inductance explained. In addition, as the device configuration is dependent on the frequency, and the frequencies may span a large range, also the optical domain is included here whereby the resonator may resemble an optical resonator.

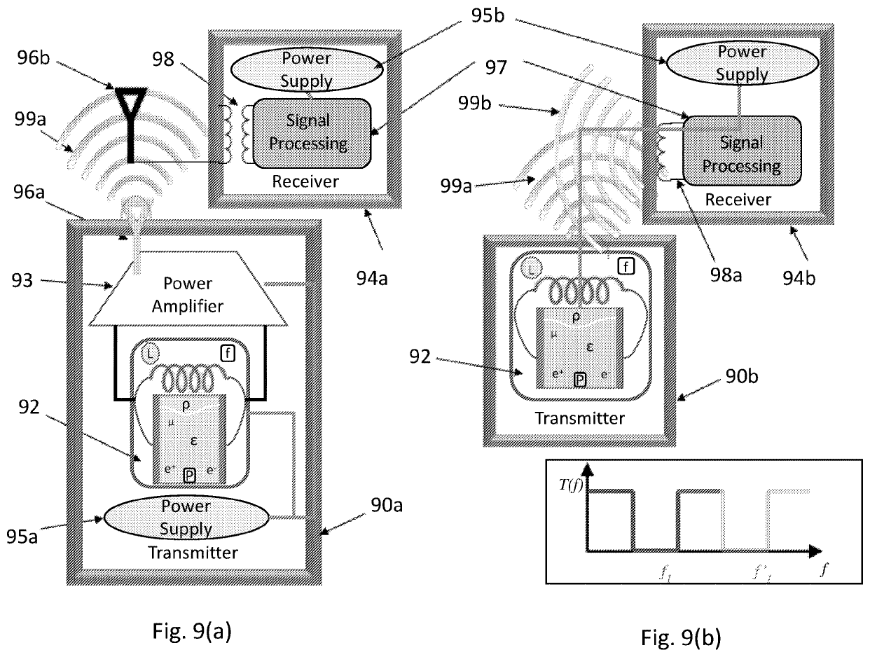

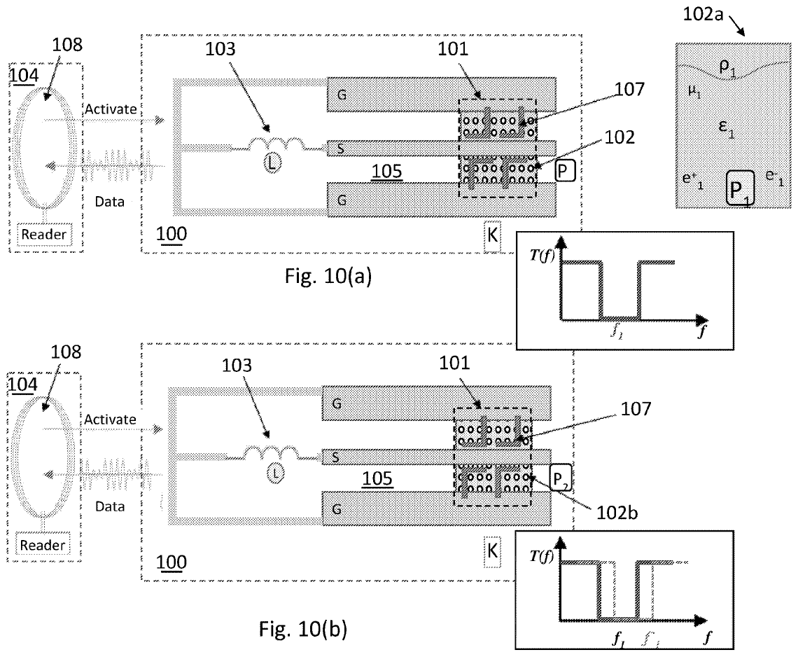

FIGS. 9A-10B illustrate generic transmitters where the sensor as described is the resonant circuit. Referring to FIG. 9A, device 90a is a transmitter. Any transmitter has an oscillator that provides EM waves and dictates the broadcast frequency. In this case, the oscillator is device 92, which is composed of an inductor and a capacitor, where the capacitor is the “sensor.” The oscillations dictated by the oscillator, in order to be able to be broadcast to a distance, need to be maintained and amplified. This task generally is assigned to some kind of amplification element 93. For all these tasks, some energy source 95a is required. The amplified EM signal is transferred to an antenna 96a, that radiates the EM waves 99a. The signal thus being emitted is received by an antenna 96b of a receiver 94a. Within the receiver, some kind of transformation or coupling 98 transfers the waves collected by the antenna, eventually, into a signal processing element 97 that amplifies, filters and processes the received signal. In this embodiment, a power supply 95a is required, as well as an electronic component forming the amplifier. This embodiment is thus useful in cases that the device is required to transmit and communicate over large distances. In a case that smaller distances are required, some components like the amplification component may be more modest or even eliminated.

FIG. 9B illustrates a second, preferred, embodiment of a transmitter. Device 90b does not include the amplification unit, the antenna or even the power supply. The theory of such devices is well known and rests in the broad category of RFID and NFC technologies. In order for device 90b to initiate its functioning, it requires some energy means. The energy source is part of the power supply 95b that resides in transmitter-receiver 94b. The power supply is transferred through the signal processor and converted into an oscillating EM wave coupled through inductive element 98a through EM field 99b to the inductor L of device 90b. The energy being collected by inductor L of the oscillator 92 will start to oscillate. The oscillation frequency of the received signal, and therefore the received power supply might match the natural resonant frequency f₁ of the oscillator 92. If the natural frequency of the transmitter-receiver and device are not equal, then the coupling of the transmitter-receiver and the device might not be optimal. Therefore, if the frequencies of both elements are not tuned, not much energy will be transferred between both. When the frequency of transmitter-receiver 94b is equal to the frequency of oscillator 92, the oscillator will enter into resonance and will draw the most energy possible from the transmitter-receiver. This can be regarded as if the device 90b through oscillator 92 is sending a signal 99a that is coupled to the coupling inductor 98a, which in turn is processed by signal processing unit 97. From a power management perspective of receiver-transmitter 94b, the coupling can be regarded as a dip in the energy return or maximum energy being drawn by the device. This is depicted in the chart at the bottom of FIG. 9B as the dip on the left side at the resonant frequency f1. This energy dip occurs when there is matching of the frequencies between transmitter-receiver 94b and device 90b.

Accordingly, the process of the detection of the sample according to this embodiment is to set the transmitter-receiver to hop or scan its transmission through one or more frequencies, through discrete or continuous values, and getting the coupling response signaled by an energy dip at the resonant frequency f1 of the device 70b. A different frequency occurs at a non-detection state f1 versus a detection state frequency f′1. The difference in frequency, and the location in frequency domain where the dip occurs, is the signaling of a non-detection versus a detection state. As understood by those skilled in the art, it is possible to scan a large range of frequencies, but also it is possible to scan only two frequencies, one for the non-detection state and one for the detection state, or even only one frequency of the detection state only, so if at this frequency there is an energy dip, it is a sign that detection has been made, otherwise no-detection occurred. It also clear that both frequencies may be very apart or different from each other or might be very close, even almost overlapping, as long as that they can be technically measured as different values.

As used in the present disclosure, whenever a dip in frequency spectrum is described, the wording should be understood as including a dip or a peak and can be used interchangeably, as long as adaptions are implemented for the appropriate effect. Therefore, wherever a dip in a frequency chart is mentioned, there might be a similar case where a peak in a frequency chart may occur. Also, although inductive coupling was mentioned in the example, additional coupling methods such as capacitive methods can be implemented as well and in fact they are exemplified in following examples as well.

FIG. 10A illustrates an embodiment of the sensing system whose principles were detailed. In this embodiment, a transmitter-reader (TR) 104 is shown with its coupling element 108 emphasized. The transmitter-reader 104 is generally similar to the transmitter-reader 94b described above in connection with FIG. 9B. For purposes of simplicity, the additional elements of the transmitter-reader are not described again here. The coupling element is illustrated as an inductor. It is understood that depending on the EM frequencies, distances etc., the details of the coupling element may change and resemble coils, or strips of metals etc. and maybe even some kind of optical device such as an optical fiber, a prism etc. or, as it is shown in this embodiment, an inductor. The coupling element 108 couples to coupling element 103 of device 100. Coupling element 103 is adapted to coupling element 108. Specifically, in this embodiment, the coupling element 103 is an inductive element, such as an inductor, with inductance L. As shown in FIG. 10A, this embodiment is illustrated as a planar technology implemented with a microstrip design. Therefore, the coupling inductor is wired to a coplanar waveguide (CPW) 105, where the signal propagates in the central conductor (S) and the outer conductors are ground (G). In such a configuration, the signal being received by the inductor 103 advances through the waveguide 105. The signal eventually arrives to sensing element 101. As explained so far, the sensing element measures the sample being tested through the material properties. The sensor 101 is a resonant element. In general, the resonator 107 (L-shaped lines) may be composed of metal geometries well known to those skilled on the art for designing resonators of this type, whose geometries define inducting strips and capacitive gaps.

The resonator 107 properties are affected by material 102 with properties P existing within and around the geometrical design of the resonant elements all together, forming the resonator 107 with resonant frequency f₁. In the configuration shown in FIG. 10A, as the resonator oscillates at resonance, it behaves as a band stop filter (BSF). A band stop filter is also known as a “stop band filter.” A band pass filter may be used in place of a band stop filter, with the appropriate technical modifications. In the case of a BSF, when a signal with the resonant frequency f₁ advances through the waveguide, the resonator behaves as a short circuit for the signal at that frequency, preventing it from proceeding and actually cutting it off. As a result, the signal is sensed attenuated at the transmitter-receiver, as it normally would be. The effect of such a short circuit on the coupled system of the transmitter-receiver 104 and devices 100 is sensed as a dip in the signal received at the transmitter-receiver, as shown in the graph of FIG. 10A, at frequency f₁. As a result, it is possible to scan through the transmitter-reader over the frequency domain and detect the dip at the sensing element identified as K, when the appropriate frequency f1 is reached.

Now, it is possible to assume that in addition to the device and the transmitter-receiver (TR) there is a sample to be tested and identified. The sample is regarded as a material 102a with parameters values defined as P1. As described above, the material is brought into sensing contact with the sensing component. And as explained, the sensing component is the band stop filter 101, composed of the inductive-capacitive elements 107 that are affected by the material properties. By bringing the sample material to the sensing area together with the existing material, an overall new material 102b is effectively considered as having properties P2. As the material properties had changed, obviously the effect of the resonator changed, thus changing the resonant frequency to f′₁ as shown in FIG. 10B. As it is shown in the corresponding chart, the frequency is shifted by some amount from the original frequency. While the receiver scans the frequency domain, the resonant frequency for sensor K now will be f′₁ instead of f₁. The receiver eventually will identify this frequency as a positive detection at the sensor K. Therefore, it is possible to monitor with the device by scanning through the transmitter-receiver over the frequency domain and detecting the dip at the sensing element identified as K, when the appropriate frequency f₁ is reached. The sensing element is identified, and it is concluded that there is no sample being detected. If instead of f₁, frequency f′₁ is sensed, sample detection is notified. Certainly, it is possible to continuously scan the frequency domain and identify any frequency dips (or peaks) to know the status of detection. Conversely, it is possible to scan some more limited number of frequencies, including but not limited to only the two frequencies involved. Yet still, for identify a signal and the status of sensor K detection, still it is possible to use only one frequency. This may be the one where the sample is involved, i.e., f′₁. If this frequency is detected, then a sample has been detected, and while no detection exists it is assumed that no sample was detected.

In this and other examples throughout the disclosure, some details of the circuits are shown, while, intentionally other elements that are obvious and well known to those skilled in the art are not represented. This presentation serves to focus and make more clearly the teachings of this disclosure. Among these elements that were not shown are elements such as terminations normally implemented in waveguides, such as shorts, loads (i.e., 50 ohm), grounding surfaces, etc.

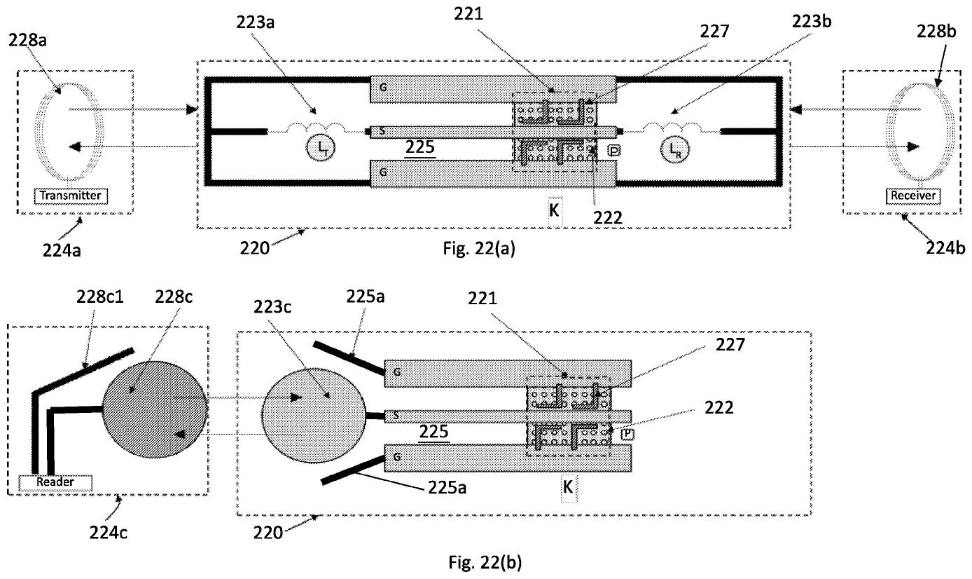

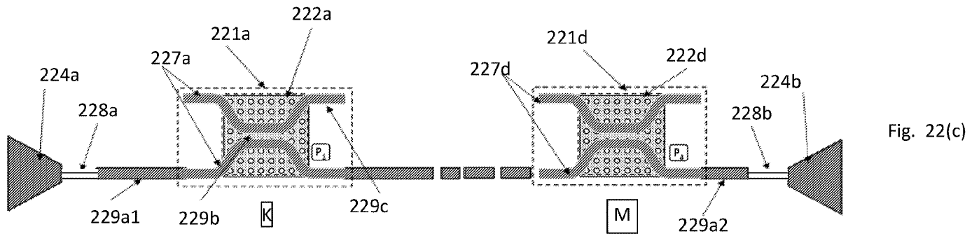

Although the coupling for reading and transmitting the signals are shown to occur at only one side/port of the waveguides, it is obvious and claimed also that the same teachings can be applied such that there is one coupling element for transmitting/receiving generally placed at one end of the waveguide side/port and a second coupling element for receiving/transmitting that is placed at other end of the WG, while the receiver and transmitter can be the same device or a receiver device and a transmitter device might be used separately. In such context with reference to FIG. 22A, sensor device 220 is shown with a waveguide 225 in which a sensor 221 (but multiple sensors may exist as well as in other examples in the disclosure) has a resonator 227 that is affected by material 222. However, in contrast to other embodiments, at each side of the waveguide/transmission line a coupling element 223 exists. At one side there is a coupling element 223a represented by inductor L_{T} which couples to transmitter 224a through coupling element 228a represented as a coil. The transmitter may either transmit a signal as previously disclosed, or, if arranged so, receive a signal, or both, as it is preferred by the implementation. At the other end of the transmission line, a second coupler 223b, represented by coil L_{R}, is coupled to receiver 224b through coupling element 228b, represented as a coil. In this case, the receiver may receive a signal sent by the transmitter or send a signal to the transmitter, or both. Using this configuration, it is possible to implement the sensor as a band stop filter (BSF), a band pass filter (BPF) or both cases as to monitor both or any of the returned or transmitted signals.

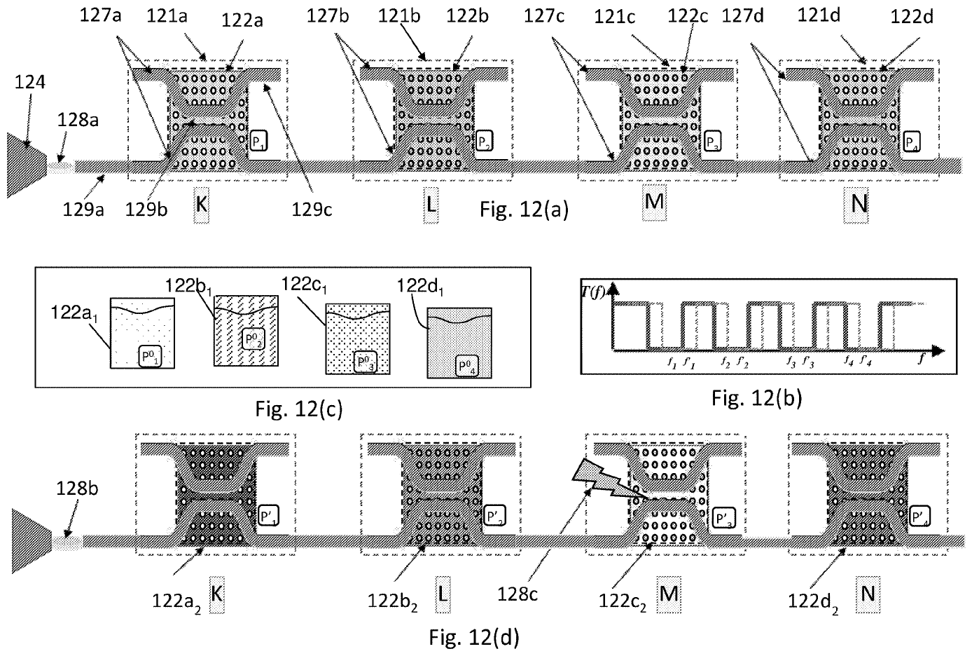

The general option to have the coupling element not necessarily at one end of the waveguide was previously exemplified through the RF EM range of the spectrum thru the use of coil like inductors. However, as discussed throughout the disclosure, the teaching may apply to other frequency bands of the EM spectrum as the couplers might take various forms accordingly. To emphasize this, if the EM waves used are in the optical range, the coupling as well can be placed at both ends of the waveguide as shown in FIG. 22C and explained in detail, while dealing with the optical spectrum examples in reference to FIG. 12.

Still, referring to FIG. 22B, although in the examples the coupling was pictured as an inductor, and generally as a coil, such kind of coupling is not the only kind of structure that might be used. In some cases, and especially at higher frequencies of the EM spectrum range, an inductive component can be realized only by a line of conductor or a structure other than a coil. In such cases, a coupling can be an inductive coupling (mainly magnetic effects), a capacitive coupling (mainly electric effects) or a combination of both (EM coupling). In such cases, the coupling elements may resemble different geometric forms. Such geometries are exemplified in, but not limited to, the representation of FIG. 22B. In FIG. 22B, device 220 is shown, where the sensor 221 with the resonator 227 affected by material 222 is placed along waveguide 225. However, the coupling element is a capacitive-inductive plate 223c. The coupling element may or may not be accompanied by respective waveguide ground extensions 225a. The coupling is done to the reader 224c through a respective coupling component 228c and possible grounding extension 228c1. Although a circular coupling element is depicted, other geometrical forms and configurations are also possible and envisioned in such cases.

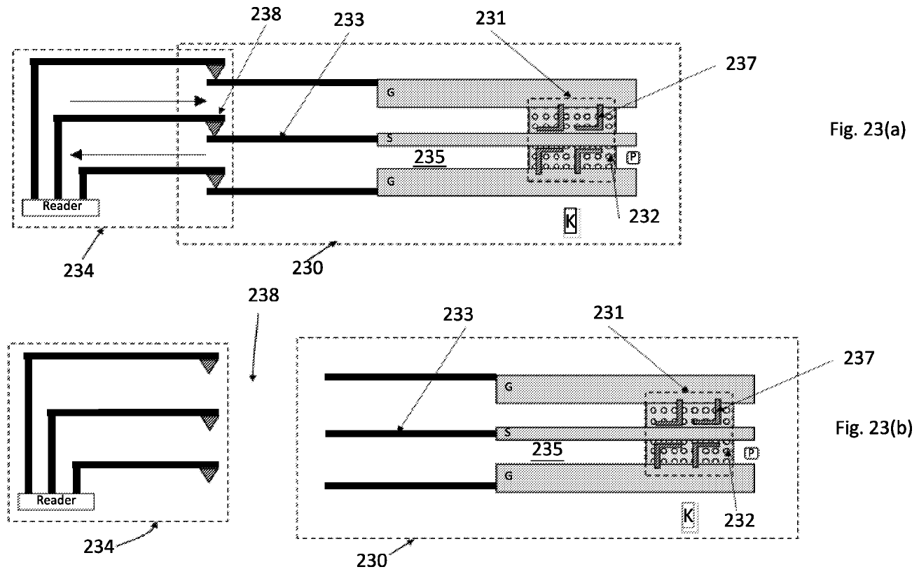

Another clarification relates to the examples given and the other embodiments shown through the entire disclosure. Although the coupling of the sensor device and the receiver-transmitter is shown and explained using EM waves, and the implementation of EM waves is at a distance (through space) i.e., “antennas” etc., the coupling and the waves are not necessarily transmitted through space, it is possible to make the same claims using direct connections between the RT and senor device. Such transmission can be done through adequate cables, such as coaxial cables, printed lines and a connector or in optical cases using fiber optic connectors, as it might be adequate for each case. In reference to FIG. 23A, a sensor device 230 similar to the disclosed in previous and later examples is shown. The device contains a sensor 231 with a resonator 237 being affected by material 232. The signals are sent through a waveguide 235. However, in contrast to other examples, the coupling to the reader 234 which may transmit or/and receive the signal, is not done wirelessly. Instead, the device contains a coupling/connecting element which in this case is a conducting extension to the waveguide which connects to the reader by a set of connecting elements that form a conductive path between the sensor device waveguide and into the reader circuit. The connection might not be rigid and can be able to disconnect as to replace the sensor device and connect a different one if so-chosen, as shown in FIG. 23B. There are many ways to implement the connection, such as element 233 being a PCB plate with lines 233 printed on them and connector 238 being spring loaded conductive connectors or any other possible connections well know in the art, such as off the shelf standard connectors between electrical components. Of course, the details of the connection depend, among other things, on the actual dimensions and the EM wave spectrum range being used.

The embodiment of FIGS. 10A-10B showed the detection of a single sample. However, as explained with reference to FIG. 3 and FIG. 4, it is preferable to be able to detect more than one sample using an additional number of sensors for each species to be detected. To do that, a kind of multiplexing-demultiplexing capability is required. The way to implement a MUX-DEMUX action in the sensor of FIGS. 10A-10B is now explained.

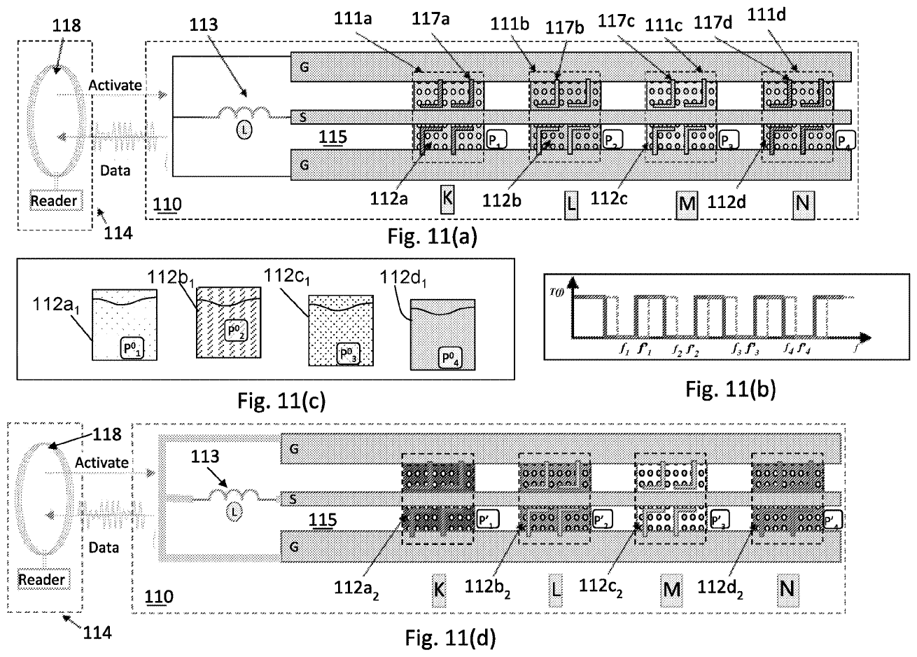

Referring to FIGS. 11A-11D, the illustrated device is similar to the ones explained previously in FIGS. 10A and 10B, with the main difference that there is more than one sensing site (111a, . . . 111d) for more than one sensing sample (112a1, . . . 112d1) to be detected. Therefore, a transmitter-receiver (TR) 114 is shown, with its coupling element realized by inductor 118. Inductor 118 couples to inductor 113 with inductance L at device 110. The signal thus received advances through the waveguide (WG) 115. However, in this embodiment, instead of reaching a single measuring element, the signal meets several elements 111a, 111b, 111c, 111d, also identified as K, L, M, N. Each sensing element has its respective resonator 117a, 117b, 117c, 117d. Each resonator may have a different configuration, such that together with the respective material 112a with properties P1, 112b with properties P2, 112c with properties P3, 112d with properties P4 and so forth as in FIG. 11A, each respective resonator has a resonant frequency of f₁, f₂, f₃, f₄, etc. Each sensing element may behave as a band stop filter (BSF). When frequencies are scanned through the transmitter-receiver, each time that the frequency of a resonator is met, a dip in the power will be sensed, as shown in the solid line of FIG. 11B, marking which of the resonators is being activated or tested. Thus, this detection effectively demultiplexes each of the sensor elements to transmit its information, i.e., its frequency, as required. It is clear to those skilled in the art, that although at a respective frequency a dip in power was exemplified, other signals may be used to indicate the resonant frequency such as a peak in power or a peak or dip in voltage/current amplitude or light intensity change or any other measurable parameter that can be coupled or used to measure the resonating effect as function of the frequency of the device sensing elements.

Referring to FIG. 11C, for the sensing of a sample agglomerate composed of materials: 112a1 with properties P01, 112b1 with properties P02, 112c1 with properties P03, 112d1 with properties P04, if they are placed in sensing contact with their respective sensing elements, the combined effect of the existing material properties with the sample material properties, as shown in FIG. 11D will change the overall material properties of each sensing element to 112a2 with properties P′1, 112b2 with properties P′2, 112c2 with properties P′3, 112d2 with properties P′4, thus changing the respective resonating frequency of respective elements to f′1, f′2, f′3, f′4, etc., as in the dotted line of FIG. 11B. Therefore, by scanning by any method as indicated previously, the frequency domain, it will possible to determine according to the frequency being detected which detector is being tested and if in the corresponding detector a sample to be tested has been identified. Thus said, if any of the f1, f2, f3, f4 frequencies had been detected it is clear that sensors K, L, M, N were identified as not detecting a sample state. On the other hand, if any or all of the frequencies f′1, f′2, f′3, f′4, were identified, this indicates that the corresponding sensor element K, L, M, N has been identified as being in positive detection of the respective sample.

In the specific embodiment of testing for DNA strands, the band stop filter is a resonant circuit including a capacitor with the single-stranded DNA between the capacitor plates. As previously discussed in connection with FIGS. 7A-7C and 8A-8C, the characteristics of the capacitors depend on the material residing between and around the capacitor plates. Here, the material between the capacitor plates is the biological material, namely the DNA-like fragment strands i.e., the cDNA/oligo ssDNA molecules. This ssDNA like material has properties such as dielectric, permeability, or resistive constants that eventually affect the BSF performance. Therefore, ssDNA-like material of some structure 112a which has properties P1 is placed in sensor 111a which causes it to have a characteristic resonant frequency f1, ssDNA-like material of some structure 112b which has properties P2 is placed in sensor 111b which causes it to have a characteristic resonant frequency f2, ssDNA-like material of some structure 112c which has properties P3 is placed in sensor 111c which causes it to have a characteristic resonant frequency f3, ssDNA-like material of some structure 112d which has properties P4 is placed in sensor 111d which causes it to have a characteristic resonant frequency f4, and so forth. When a frequency is sent by the transmitter-receiver 114 and is coupled into the device 110 by coupling element 113 propagating through waveguide 115, then if the frequency matches the resonant frequency of any of the sensors/filters, the impact of the resonance will be sensed by the transmitter-receiver and seen as a dip in the intensity of the signal as shown in the chart of FIG. 11B, for frequencies, f₁, f₂, f₃, f4 for sensors K, L, M, N (assuming that it was designed such that each filter has a different frequency). Knowing which ssDNA-like material was placed in which filter and which frequency belongs to which filter, it is possible to identify at any moment which filter is being interrogated.

Referring to FIG. 11C, for the sensing of a sample agglomerate composed in this case of ssDNA-like materials: 112a1 with properties P01, 112b1 with properties P02, 112c1 with properties P03, 112d1 with properties P04. In FIG. 11D, the fragment to be tested is brought into the testing zone, where ss-DNA-like materials 112a, 112b, 112c, 112d are located. If the fragment matches the present ssDNA-like material, they hybridize forming a new dsDNA fragment-like molecule. This new molecule will have different properties. Thus: dsDNA-like material of some structure 112a2 which has properties P′1 is created in sensor 111a which causes it to have a characteristic resonant frequency f′₁, dsDNA-like material of some structure 112b2 which has properties P′2 is created in sensor 111b which causes it to have a characteristic resonant frequency f′₂, dsDNA-like material of some structure 112c2 which has properties P′3 is created in sensor 111c which causes it to have a characteristic resonant frequency f′₃, dsDNA-like material of some structure 112d2 which has properties P′4 is created in sensor 111d which causes it to have a characteristic resonant frequency f′₄ and so forth. As now the characteristic resonant frequency has changed, the response detected by the transmitter-receiver will show dips in frequencies f′₁, f′₂, f′₃, f′₄ different from before for sensors K, L, M, N. From this difference it is possible to deduce which sensor is being inspected and also if the signal is of the sensor in its base state with ssDNA, or in its hybridized state dsDNA. In the latter case, it means that the specific fragment was detected. It is clear to those skilled in the art that at any moment not all the sensors must necessarily show positive detection, since the fragment to be detected may or may not be present in the sample agglomerate. However, the frequency shift is an indication of the detection state.

As the frequency might be affected by the amount of the fragments being hybridized, the frequency shift can be an indication of the amount of the material in the sample to be tested. For example, suppose that between the capacitor plates there are 1000 ssDNA strands. They have some dielectric constant value which represent them all together. Now, if 100 of these strands had combined into ds DNA, then there are 900 having one dielectric constant and 100 having a different dielectric constant. The effective dielectric constant will be the effect of both, and therefore the frequency will shift according to the combined effect. This change will shift as more and more molecules of the 1000 will be hybridized until all the molecules are transformed into ds. So, there will be a shift between the frequency of the SS into the frequency of the DS. So, by knowing the frequency between these two limits, it is possible to infer the amount of material that was hybridized.

Many ways of testing the frequency domain are possible. It is possible to scan all the possible frequencies in range and detect the dip/peak at each relevant frequency, but it is also possible to scan only frequencies f1, f2, f3, f4 and f′1, f′2, f′3, f′4 or even only frequencies f′1, f′2, f′3, f′4.

The details of the actual implementation of such systems as described may vary according to the frequency. As such, elements that have been mentioned previously may receive different forms accordingly. For example, the reader-transmitter and its coupling element may have a different configuration depending on the frequency. In the RF domain, at low frequencies, it may resemble a solenoid, while for higher frequencies a conducting line having some inductance might be sufficient. In such cases, for low frequencies, the coupler may behave more like a transformer and the waveguide will appear just as a simple connecting conducting line.

On the other hand, at optical frequencies or even higher frequencies the transmitter might be some light emitting (e.g., a LED or light bulb) and receiving (e.g., a photodiode or phototransistor or fiber optic cable) component as known in the art, and the device configuration also may change accordingly. Therefore, at optical frequencies the receiver may contain some optical coupler and the waveguide may take the form of some type of fiber optic.

FIGS. 12A-12D schematically illustrate an embodiment of a transmitter-receiver which is based on a planar fiber optic system. In FIG. 12A, a transmitter-receiver (TR) 124 is depicted. In this example, the EM frequency is in the optical domain. The TR couples, sends, and receives a light beam 128a through a respective light coupling element into an appropriate optical waveguide, such as an optical fiber. In FIGS. 12A-12D a planar fiber optic system (as seen from above) is schematically drawn. The light 128a from the transmitter-receiver (TR) 124 enters an optical fiber, namely the waveguide (WG) 129a. The light advances through the fiber and can either exit from the other end and/or be reflected within the fiber optic (WG) element. As mentioned earlier for other examples, the figures contain only elements and components that support clear understanding of the description. Other components were intentionally disregarded (such as terminations, reflective coatings etc.), which are all well known to be used as needed by those skilled in the art. The light passing through the waveguide or fiber optic may be coupled to another fiber 129c (partially shown) that may lead to another optical circuit (not shown). The coupling between the fibers is done by having them closely spaced, with a space 129b, which allows the light to transfer/tunnel from one fiber to the other. This kind of optical planar coupler 127a is well known in the art and is used among other to design planar fiber optic Mach-Zehnder interferometers, for example. The properties of the light coupling performance of the coupler 127a are dependent on the gap 129b, geometrical dimensions, as well as the material properties of the material 122a in the vicinity of the gap 129b. As a result, changing the properties of the material will change the frequency of the EM wave/light that will be transferred from path 129a to 129c, transforming the coupler 127a into an optical version of the sensor. Therefore, sensor 127a which depends on material 122a with properties P1, becomes the optical version of measuring component 121a identified as element K. As in previous embodiments, additional sensing components may be placed along the waveguide 129a, creating in general sensing components 121a, 121b, 121c, 121d etc., with sensing elements 127a, 127b, 127c, 127d etc., depending on materials 122a, 122b, 122c, 122d etc. with properties P1, P2, P3, P4 etc. for devices K, L, M, N . . . that will remove, from the input light beam spectrum in the fiber, the “colors” corresponding to frequencies f₁, f₂, f₃, f₄. Therefore, the reflected/transmitted/received light at the transmitter-receiver 124, will miss those frequencies and indicate a dip (or a peak) in the received light spectrum at those frequencies as sketched by the solid line of the chart of FIG. 12B.

Referring to FIG. 12C, a material agglomerate sample is composed of materials 122a1 with properties P01, 122b1 with properties P02, 122c1 with properties P03, and 122d1 with properties P04. When these materials are placed in sensing contact with their respective sensing elements, the combined effect of the existing material properties with the sample material properties, as illustrated in FIG. 12D, will change the overall material properties of each sensing element to 122a2 with properties P′1, 122b2 with properties P′2, 122c2 with properties P′3, 122d2 with properties P′4, thus changing the respective coupling frequency of respective elements to f′1, f′2, f′3, f′4, etc., as in the dashed line in FIG. 12B. Therefore, by sending or scanning a light beam 128b with the relevant frequencies of the frequency domain, it is possible to determine according to the frequency being detected, which detector/sensing component is being tested and whether in the corresponding detector a sample to be tested has been identified. Thus, if any of the f1, f2, f3, f4 frequencies are detected, it is clear that corresponding sensors K, L, M, N are in a state of not detecting a sample. On the other hand, if any or all of the frequencies f′1, f′2, f′3, f′4, are identified, this indicates that the corresponding sensor element K, L, M, N is in a state of positive detection of the respective sample.

At this point a matter should be clarified as it was done for the EM RF case above, in reference to FIGS. 22A-C and 23A-B. Throughout the illustrated examples, the reader 124 in FIG. 12 is shown at one end of the fiber 129a of the sensor device for which the light signal can be sent and received from the same side/port. The teachings of this disclosure also teach that a transmitter receiver such as 124 can be placed in addition at the other end of the fiber and it, as well, can send or receive optical signals. Therefore, it is possible to have a transmitter at one side and a receiver at the other side or any combination of both such that it will be possible to monitor not only the returning signal but also the passing signal that arrives to the other end, regardless from which side the signal originated and even from both. This can be seen in reference to FIG. 22C, where a first coupling element 224a at one end of the optical waveguide is intended to send or receive or both a light signal 228a which enters or exits or both from optical fiber 229a1, similar to the explanations of FIG. 12. The light signal advances through the waveguide and meets one or more sensors 221a, . . . , 221d, marked as K, . . . , M as explained previously. In it, the light passing through the waveguide or fiberoptic may be coupled to another fiber 229c (partially shown) that may lead to another optical circuit (not shown). The coupling between the fibers is done by having them closely spaced, with a space 229b, which allows the light to transfer/tunnel from one fiber to the other. This kind of optical planar coupler 227a, . . . , 227d is well known in the art and is used among other to design planar fiber optic Mach-Zehnder interferometers, for example. The properties of the light coupling performance of the coupler 227a, . . . , 227d are dependent on the respective gap 229b, geometrical dimensions, as well as the material properties of the material 222a, . . . , 222d in the vicinity of the respective gap 229b. As a result, changing the properties of the material will change the frequency of the EM wave/light that will be transferred from path 229a to 229c, transforming the coupler 227a, . . . , 227d into an optical version of the sensor. Therefore, sensor 227a, . . . , 227d which depends on material 222a., 222d with properties P1 . . . , P4 becomes the optical version of measuring component 221a, . . . , 221d, identified as element K . . . M. However, in this case, at the other end 229a2 of the fiber optic waveguide, a light signal 228b may couple to an additional coupling element 224b that can receive or transmit or both an optical light signal through the sensor.

It is also emphasized that although in the example of FIG. 12, the light coupling is shown as the light signal 128a transits the gap through space wirelessly, the same teachings are valid for a case when the fiber optic is physically connected to the receiver transmitter end by any light waveguide connecting method, including off the shelf available connectors for such cases as are well known by those skilled in the art.

In the specific embodiment in which the material is a biological material and in particular a DNA-like fragment, the material in the couplers zone in general and between the fibers/WG in particular is the biological material, namely the DNA-like fragment strands i.e., the cDNA/oligo ssDNA molecules existing between the coupler fibers. This ssDNA like material has properties such as dielectric, permeability or resistive constants that eventually affect the couplers' performance. Therefore, ssDNA-like material of some structure 122a which has properties P₁ is placed in sensor 121a which causes it to have a characteristic coupling frequency f₁, ssDNA-like material of some structure 122b which has properties P₂ is placed in sensor 121b which causes it to have a characteristic coupling frequency f₂, ssDNA-like material of some structure 122c which has electrical properties P₃ is placed in sensor 121c which causes it to have a characteristic coupling frequency f3, ssDNA-like material of some structure 122d which has properties P₄ is placed in sensor 121d which causes it to have a characteristic coupling frequency f4, and so forth. When a light beam of a frequency/color is sent by the transmitter-receiver 124 and is coupled into the device 120 through light beam 128a propagating through waveguide 129a, then if the frequency/color matches the frequency of any of the sensors/couplers, the impact of the coupling will be sensed by the transmitter-receiver in general and will be seen as a dip in the intensity of the signal, as shown in the chart of FIG. 12B, for frequencies, f₁, f₂, f₃, f₄ for sensors K, L, M, N (assuming that it was designed such that each coupler has a different frequency). Knowing which ssDNA-like material was placed in which coupler and which frequency belongs to which coupler, it is possible to identify at any moment which coupler is being interrogated and which material is being interrogated.

The ss fragment to be tested is brought into the testing zone where the ssDNA-like material resides. If the fragment matches the present oligo, they hybridize forming a new dsDNA fragment-like molecule. This new molecule will have different electrical properties. In reference to FIG. 12D, this means that new materials are present at the sensing zone, namely: dsDNA-like material of some structure 122a2 which has properties P′1 is created in sensor 121a which causes it to have a characteristic frequency f′1, dsDNA-like material of some structure 122b2 which has properties P′2 is created in sensor 121b which causes it to have a characteristic frequency f′2, dsDNA-like material of some structure 122c2 which has properties P′3 is created in sensor 121c which causes it to have a characteristic frequency f′3, dsDNA-like material of some structure 122d2 which has properties P′4 is created in sensor 121d which causes it to have a characteristic frequency f′4 and so forth. As now the characteristic frequency/color has changed, the response detected by the transmitter-receiver will show dips in frequencies f′1, f′2, f′3, f′4 different from before for sensors K, L, M, N. From this difference it is possible to deduce which sensor is being inspected and also if the signal is of the sensor in its base state with ssDNA, or in its hybridized state where the dsDNA is in the sensor's sensing area. In the latter case it means that the specific fragment was detected. It is clear to those skilled in the art that at any moment not all the sensors must show positive detection for all the available sensors, as the fragment to be detected may or may not be present in the sample agglomerate. However, the frequency shift is an indication of the detection state. As the frequency might be affected by the amount of the fragments being hybridized, the frequency shift can be an indication of the amount of the material in the sample to be tested, as discussed above in connection with FIGS. 11A-11D. As previously, discussed, many ways of testing the frequency domain are possible. These include scanning or emitting all the possible frequencies in range and detecting the dip/peak at each relevant frequency. It is also possible to scan only frequencies f1, f2, f3, f4 and f′1, f′2, f′3, f′4 for response or even only frequencies f′1, f′2, f′3, f′4 as in the single sensor element case.

Advantageously, when testing in optical frequencies, the interrogation of the sensors can be performed in parallel and not only by scanning, since through the optical fiber/WG all the frequencies/colors can be sent at the same time (i.e. white light). This is in contrast to the RF frequencies described in FIGS. 10A-10B and 11A-D, where transmitters can send a single or few frequency signals only and scanning is the method of choice for transmitting the RF signal. Therefore, for an optical device a beam of all the relevant frequencies/colors may be sent as a single beam and then interrogated at once by a simple spectrum analyzer (for example a prism).

It is also a feature of this embodiment, that for some material in general and for DNA-like labeled molecules or fluorescent and similar materials in particular, the light beam sent through the fiber and coupled to the output fiber either through the base material or the base material combined with the material to be tested, may induce a secondary light emission 128c instead or in addition to the interrogating details already explained. For example, the secondary light transmission may be caused by fluorescence or phosphorescence. This additional generated light 128c can be monitored similar to the coupled light beam or by any other method as to get either an identification signal or even additional information from the sample, beyond the basic information already disclosed.

Although an embodiment with a planar fiber waveguide optical coupler was used as an example, other ways are also possible, such as a coupling prism placed above, adjacent to, or otherwise near the input fiber in order to extract the frequencies.

FIGS. 13A-15C and 16A-1 address certain implementations of the coupling of the ss-DNA segments to the substrates.

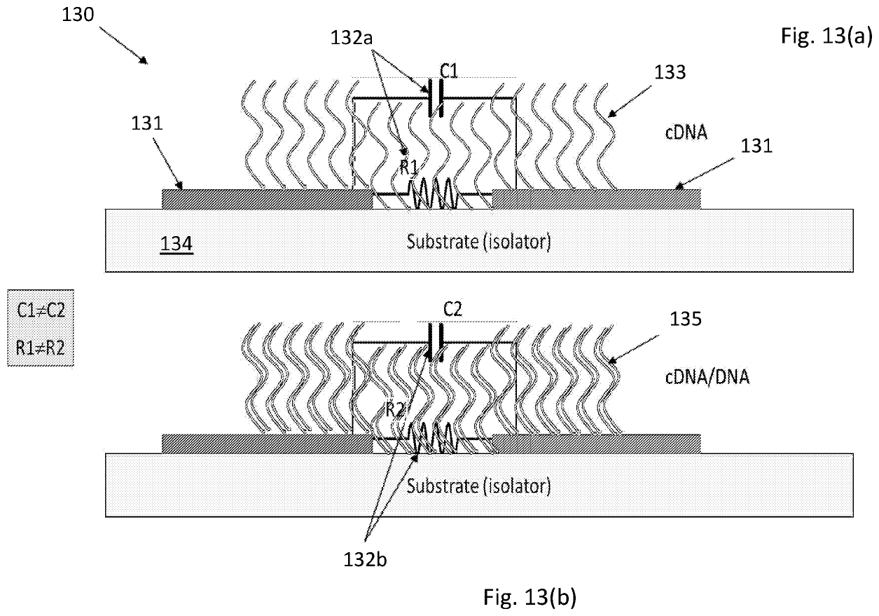

FIGS. 13A-B illustrate a specific implementation with respect to the active zone of the sensor, whether it be a capacitor or a coupler of an optical waveguide. FIG. 13A shows a zoomed detail of the sensing device 130, optionally on a substrate 134, and on it, representing the active zone of the sensor, the conducting plates 131 of the capacitor element in the case of a BSF. The illustrated device 130 may also represent the WGs 131 of the coupler at the tunneling zone in the case of a light frequency range. In either case, the oligos/DNA-like single strand fragments 133 are attached to the surface of plates or waveguides 131, either on top of it or/and in the vertical faces or/and in the gap between the plates or waveguides 131. In the example of conductive plates, due to the biological material properties, a capacitance C1 and probably some resistance R1 and other properties will get some values 132a, which eventually will impact the response frequency of the sensing element. In this respect, as shown in FIG. 13B, a DNA-like fragment might eventually bond to the already residing ssDNA-like fragment forming a new double strand DNA-like molecule 135.

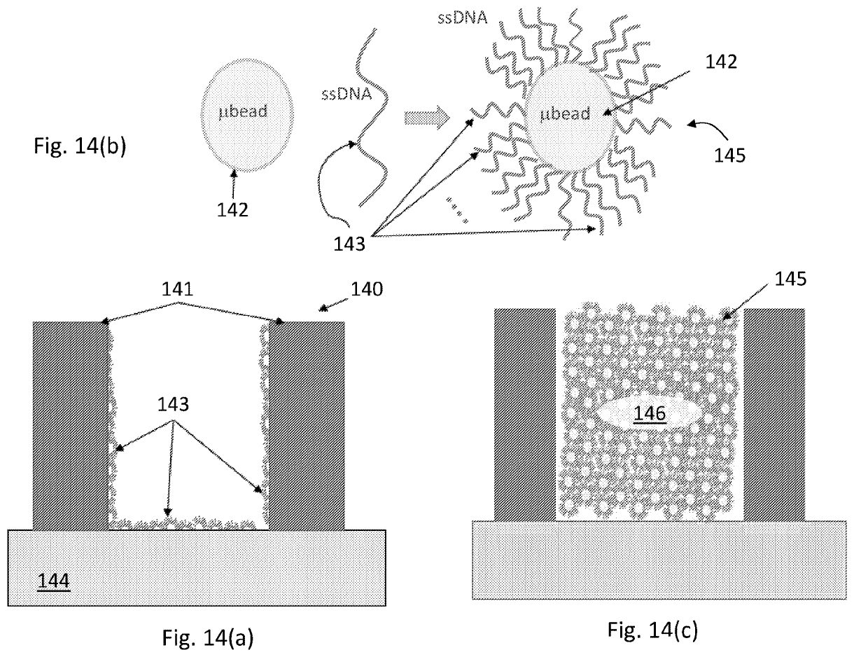

However, practical considerations such as due to fabrication or other limitations and due to the small amount of target molecules that might be available, place boundaries to the effect of the ss or ds molecules on the sensor, which might not be as pronounced as might be preferred. In this respect, as referring to FIGS. 14A-C, these challenges are addressed as to enhance the capabilities of the concept being disclosed.

FIG. 14A shows the active sensor area of a device 140. The device may have a substrate 144. In this example, a cross section side view of the plates of a capacitor 141 is shown. The plates 141 may have a different configuration, and be made of various materials including optical fibers. What it is apparent in the figure is that there is obviously some gap between the plates 141. In this case, the gap is filled with some dielectric material that may be vacuum or air or a fluid or any other substance. The gap distance affects the capacitance and obviously has some practical limit. Between the plates, the biological specific “decoding” base molecules are attached as explained previously. Specifically, ssDNA-like molecules 143 may be attached at the substrate between the plates and/or on the faces of the electrode plates (by prior art methods), as shown, and/or in any other position around the plates including but not limited to the top of the electrodes, external faces of the electrodes, at the external zone of the capacitor on the substrate, not shown, as exemplified in FIG. 14A. The amount of molecules present and affecting the dielectric constant of the capacitor in such a configuration might not be substantial enough to induce as a strong response of the sensor as might be desirable.

To enhance the effect, a method to enhance and increase the impact of the biological material is suggested, by exposing the capacitor plates to additional molecules. To this end, in FIG. 14B, a tiny bead of dielectric material with known properties 142 is used as an attaching place for the ssDNA-like molecules 143. By attaching (by prior art methods) the molecules to the tiny beads, a microbead complex 145 is created, where one or many molecules are possibly attached to each bead surface. The complexed microbeads 145 are placed between the capacitor plates as shown in FIG. 14C, presenting a more intense material 146, from the capacitor point of view, composed of the beads covered by ssDNA-like molecules. This has the effect of presenting additional molecules to the capacitor plates and increasing the exposure area on which the target molecules can bind to create the ds molecules, therefore enhancing the effect of the biological material. The beads may be of any suitable size, such as the micron scale size or even the nanoscale size. The beads may be of spherical form, but they may be made of oval, rectangular, pyramidal or any other geometrical configuration. In addition, the beads may be made of various materials including glass, polymers, ceramics, metals, magnetic material or any other material that fits the implementation. It is possible to use even beads made of DNA material itself compacted to form an agglomerate or cluster of DNA material, which can be considered also as a bead, for example by way of a compaction oligos. In general, the beads may be made hollow or may be formed of several layers of various materials or any other combination.

As explained previously, the frequency shift is dependent also on the amount of material present. In particular, the frequency is dependent also on the amount of material present on the bead surface. Therefore, it is possible to place on a single bead two or more types of ssDNA, each that combines to a specific different ssDNA to be tested. Thus, by knowing the relative ratio between the base ssDNA molecules on top of the bead surface, it is possible to understand which of the molecules are attached to the beads or both, and this from the frequency shift, which depends on the amount of material present.

For example, suppose that, initially, two types of ssDNA are placed on a bead surface. These ssDNA molecules are included at known ratios, such as 20% for type “A,” and 80% for type “B.” The corresponding resonance frequency of the sensor is an outcome of the presence of both types of ssDNA molecules. When the material to be tested is introduced, it may attach to one of the base ssDNA molecules, in the available quantities. This causes a change in the resonance frequency, which is proportional to the amount of material that is matched. Thus, if material “A” matched, the change will be less dramatic, and if material “B matched, the change will be more dramatic. In this manner, a single sensor may be used to test for presence of more than one material.

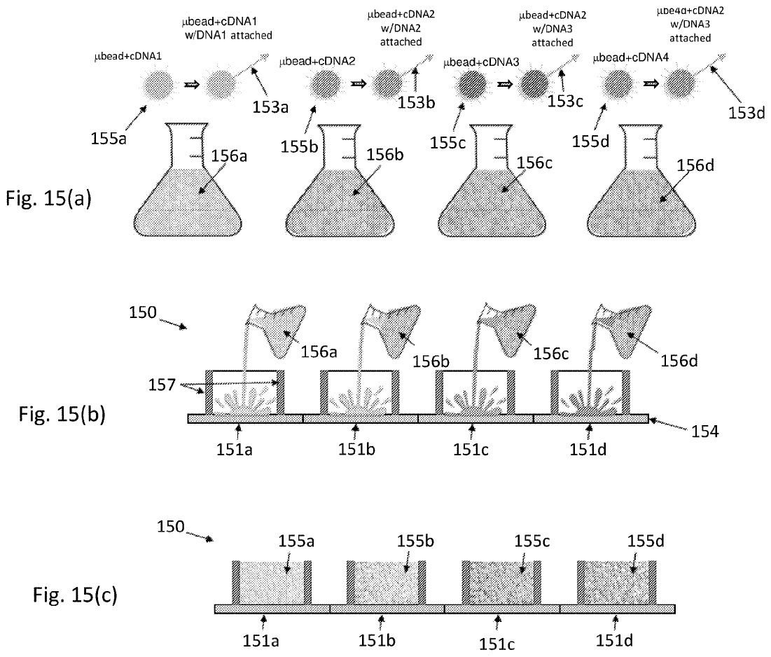

Referring now to FIG. 15A, it is possible to prepare various supplies 156a, 156b, 156c, 156d, etc., of complexes of beads. Each such supply may or may not have the same composition as the others. In each supply, such as 156a, microbeads complexes such as 155a are stored such that the complexes have microbeads with a specific “decoding” base molecule attached to their surfaces. Such specific molecules are intended to bond a specific target molecule 153a. One such bond is shown in FIG. 15A. The same goes for supply 156b, 156c, 156d and so forth made of microbeads complexes 155b, 155c, 155d etc., which are intended to bond to target molecules 153b, 153c, 153d respectively. Having a detecting device 150, as shown in FIG. 15B, with possibly a holding substrate 154 with several detecting sensor elements 151a, 151b, 151c, 151d, with its respective capacitor plates 157; the microbeads complexes material 156a, 156b, 156c, 156d is placed from the supply into the corresponding sensing areas of the capacitor in general, and in the gap between the capacitor plates in particular. Therefore, as shown in FIG. 15C, each sensing element 151a, 151b, 151c, 151d has its respective microbead material complex 155a, 155b, 155c, 155d which is intended to bond and thus detect a specific target species 153a, 153b, 153c, 153d.

One of the purposes of the use of beads is to expose additional surface area on which the ssDNA is able to attach to a surface and by this to increase the amount of material present between the capacitor plates (for example). As mentioned, the beads may be made of many materials. If the beads are conductive, such as made of metals, an additional enhancement, beyond the already disclosed, is possible as will be described.

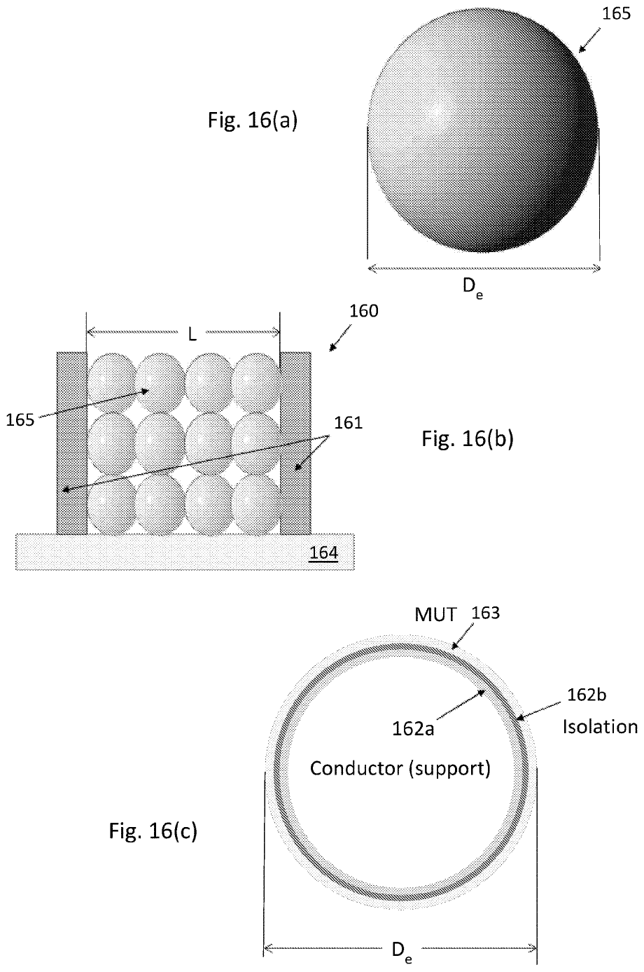

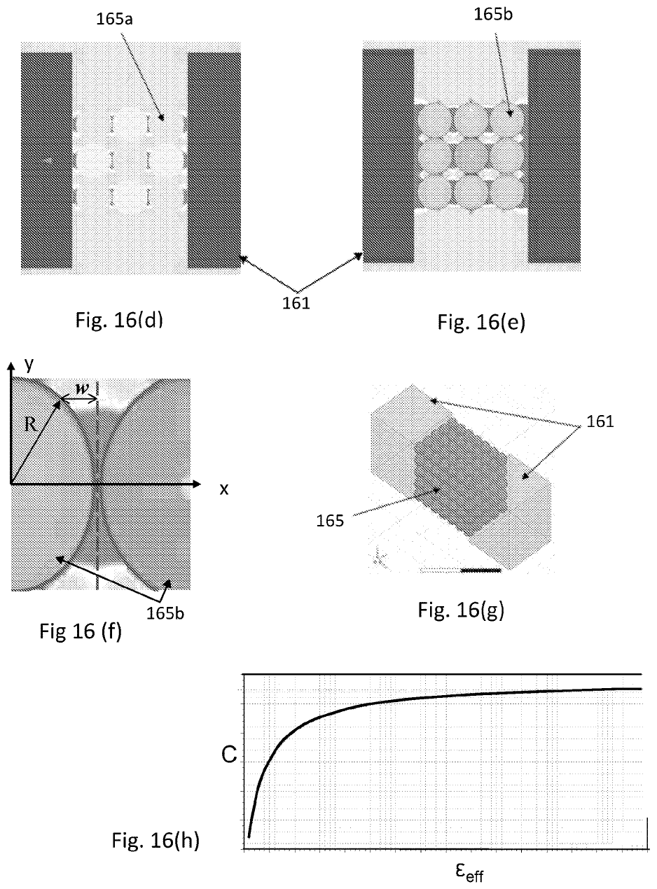

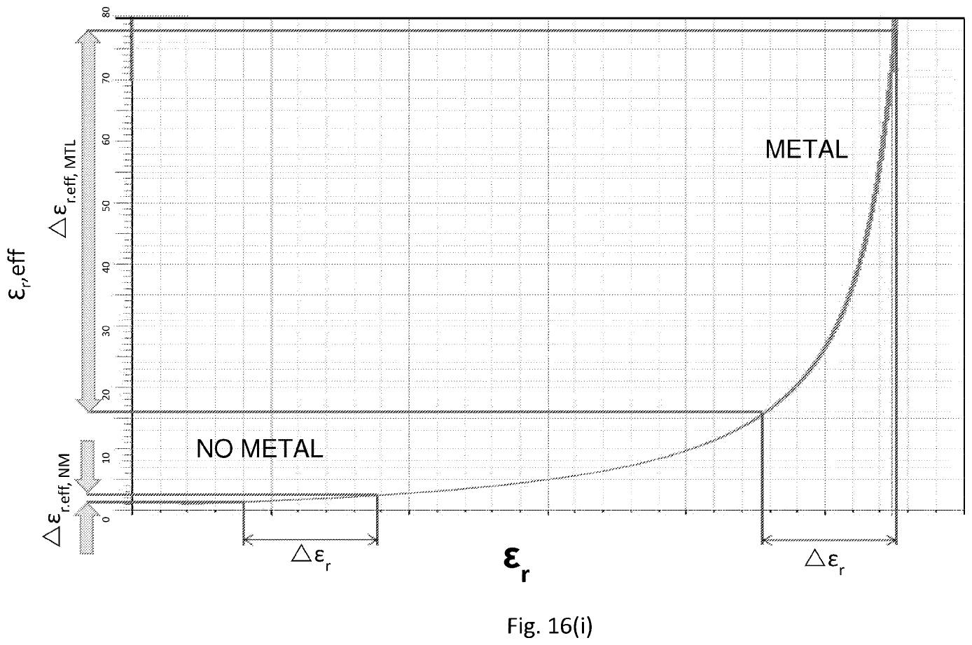

A preferred embodiment related to conductive beads is exemplified in FIGS. 16A-B. In FIG. 16A, a single bead 165 is shown. The bead has a diameter Dₑ. As shown in FIG. 16B, the beads are packed between elements of the resonator at the active zone of the sensor. To better exemplify this embodiment, the effective zone of device 160, optionally supported by substrate 164, includes two capacitor electrodes 161, separated by a distance L. As mentioned, the effective zone is filled with beads 165 which may be spheres or any other shape, either all equal in size or of various sizes, and packed at random or as space ordered possible arrangements. For ease of explanation, in this example, the beads are same size and are ordered in a 3D array with a simple cubic packed arrangement as shown in FIG. 16B.

The capacitance of a capacitor is:

$$C = \frac{{ε}_{0} {ε}_{r} A}{d}$$

wherein C is the capacitance, ε₀ is the dielectric constant of vacuum and εᵣ is the relative dielctric constant of the material between the plates, A is the surface area of the plate and d is the distance/gap between the plates. From this it may be concluded that in order to increase the capacitance, it is necessary to reduce the distance between the plates. However, if for some reason (such as fabrication limitations) the distance d is bounded to a gap L such that d=L (FIG. 16B), then the capacitance and therefore the sensitivity will have a bounding limit. Therefore, if the sensing capability were improved by placing more DNA fragment strands on microbeads instead of placing them on the surface of the electrodes only; then placing many beads will cause a corresponding limitation, since the increase in the gap d=L reduces the capacitance for the same area. So, a way to reduce the effective gap between the electrodes while still keeping the beads is required. This understanding is valid for any capacitor regardless if it is to be used as a sensor as in this disclosure or not or if the beads are covered by DNA fragments or not.

In advantageous embodiments, this challenge is met through use of metal beads. Referring to FIG. 16D and FIG. 16E, a simulation of a small portion of capacitor plates with beads distributed between them is shown. The gradations in shading are representative of the electric field strength. The beads 165a of FIG. 16D have a thin dielectric shell only; while the beads 165b of FIG. 16E have a metal shell. It can be seen a priori that there is a difference between the two cases. In the first case, the field penetrates the dielectric shell, passes inside the bead, exits from the other side of the bead and so on until reaching the second electrode. However, in the case of metal beads, there is no field inside the conductive sphere. This means that effectively, the space inside the spheres is taken out (disregarded, for all practical purposes) of the total spacing between the electrodes. In other words, if in the dielectric sphere case the entire gap and all the dielectric material within it is used for capacitance calculation; in the conductor case, only the space between (outside) the spheres should be considered and the inside of the spheres would not be taken in consideration. This effectively reduces the gap d between the capacitor plates from L to an effective value which is smaller than the diameter of the spheres multiplied by the number of spheres. In FIG. 16F, there is a closeup of two consecutive conductive beads (half of each bead is shown) where the fields between the beads are shown. It is possible to understand that if instead of beads there had been placed similar scale size cubes or bricks, then the actual spacing for the capacitance calculation would be the gap between the brick faces. In the context of spheres, the gap varies from a very small gap to larger and larger gaps as the distance increases from the center in the |y| direction. So, the calculation in this case is more elaborate, but eventually it is possible to evaluate a value for the effective gap between the spheres if replaced by a brick bead. This value is found to be very close to twice the diameter of the beads. This signifies that it is possible to reduce the effective gap by almost a factor of 2 and therefore increase the capacitance by an order of almost 2 for the same overall distance between the plates by using conductive beads. This signifies a large improvement in the sensitivity of the sensor thru using conductive beads.

The conductive beads as mentioned may be made of any conductive material including metals, conductive polymers, conductive crystals or superconductive materials. They may be made as a solid bead or as a hollow bead, and they may be covered with an isolating layer (full or partial) to prevent them from shorting between beads in contact with them. The conductive beads may be covered with a dielectric or other material. This other material may be the material to be tested, such as the ssDNA fragments discussed above. Such a bead embodiment is shown in FIG. 16C, where in this case a hollow conductor core 162a is shown. In the shown embodiment, the conductive core is strong enough to support the bead structure. However, in other cases, where the conductive layer is too thin to support the bead structure, it is possible to fill the inside of the conductive shell with one or more materials such as glass or polymers in a layered fashion or a solid fashion and even to fill all the inside of the bead with a solid conductive material, which may be the same or different from the conductive layer 162a. On top of the conductive layer may be placed an isolation layer 162b, that fully or partially covers the conductive layer to prevent a short-circuit between the bead's conductive layers. This layer may be used also as a base for the attachment of the ssDNA fragments 163, which is the material under test (MUT).

In addition, using the conductive beads, additional sensitivity improvement may be achieved through an additional effect. Just to understand the scale involved, the diameter of the beads might be on scale of Dₑ=1 μm or less. As a large number of beads are used, then for practical evaluation it is desirable to find an effective dielectric constant of the bulk of spheres. A practical way to do this is to calculate in a known way, such as a simulation, the capacitance of an array of micro beads with the required bead composition, as shown in FIG. 16G. Alternatively, it is possible to calculate the capacitance of a capacitor of a similar configuration, where instead of the beads a homogeneous material with known dielectric constant is used. This dielectric constant is referred as an effective dielectric constant, as will become apparent infra. For the last capacitor, it is possible to draw a curve of capacitance versus effective dielectric constant, as shown in FIG. 16H. By matching the value of the capacitance of the bulk beads to the respective capacitance in the curve, it is possible to define an effective dielectric constant that may be used instead of the actual dielectric material on the face of the metal beads.

By using this methodology, it is possible to have a chart that connects between the true dielectric constant of the spheres and the effective capacitance to be used for capacitance calculations in the resonators, as shown in FIG. 16I. As shown in the chart, for the same change in actual dielectric constant, Δε_{r′} the beads made of a dielectric material are in the shallow part of the chart therefore causing a small change in effective dielectric constant, Δε_{r.eff, NM}. Conversely, for the same actual difference in dielectric constant, Δε_{r′} for the example of metal beads, the change of effective dielectric constant is in the steep zone of the chart, causing a large change, Δε_{r.eff, MTL}, which means an increase in sensitivity of dielectric constant variation for the case of conductive beads. Since the change in dielectric constant is translated to frequency change in the sensor, then this increases the sensitivity of the sensor.

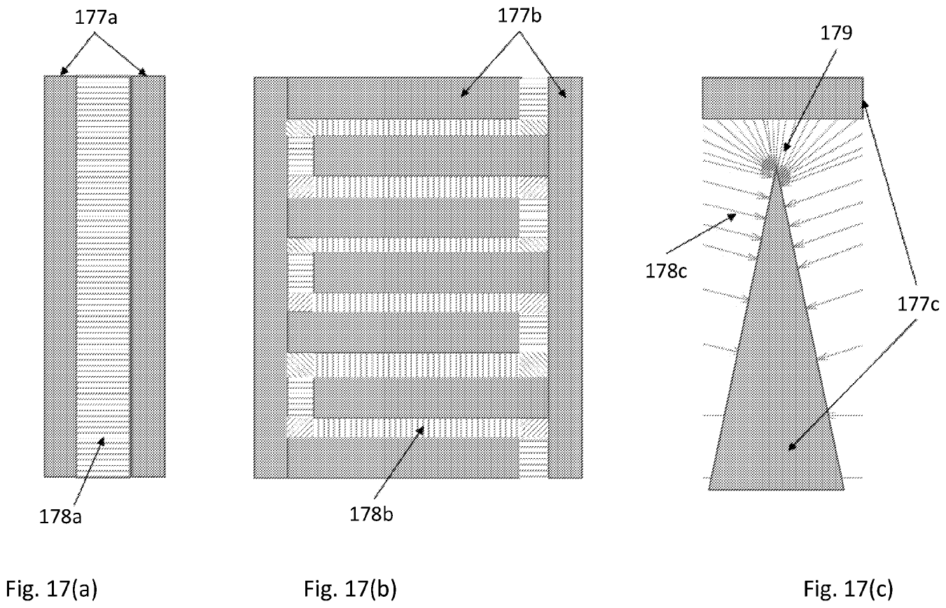

Still additional enhancement options may be used such as shown for FIGS. 17A-C, regarding additional geometrical configurations of the capacitor electrodes. In FIG. 17A, a top view of capacitor plates 177a is illustrated. This is a parallel plate capacitor geometry, where two parallel conducting plates are positioned in parallel to each other. Lines 178b represent the parallel electric field between plates, where in most of the cases the dielectric material will be put, as explained previously. Larger capacitances and better response to dielectric material changes, however, might be achieved through the use of the configuration of FIG. 17B, where the geometric layout of electrodes 177b resembles comb fingers interlaced between each other. With this configuration it is possible to receive larger effective areas and, in some cases, the impact of the dielectric material on the capacitance may be improved, if similar geometrical restrictions for all the cases apply. Still, a third case is exemplified in FIG. 17C. FIG. 17C shows an electrode configuration 177c, having the form of an apex near a plate. With this geometry, the electric field 178c concentrates and becomes higher at the apex. In cases where small amounts of target material are expected, placing the specific “decoding” base molecule at zone 179, where the field is highest, will enhance the impact of the dielectric material and therefore the detection capabilities. It is clear that those skilled in the art may find additional configurations and the examples shown in FIGS. 17A-C may represent a small differential part of a more complex geometrical configuration that contains any one of the examples above by themselves or in any combination thereof. For instance, the capacitor depicted in FIG. 17A may be a section of a ring capacitor, thus the electrodes represent the inner and outer electrodes, or it may represent a part of an S or L or other geometrically shaped configuration of the electrodes. The same thinking applies to the example of FIG. 17B, where the representation of the figure may be a differential part of a larger and more complex geometry such as a circle, a S or L or other geometry shape and the same thinking goes for the example of FIG. 17C. As mentioned previously, this complex geometry may have parts that are linear as FIG. 17A, and then become more complex as in FIG. 17B, and in specific zones have apex electrodes as in FIG. 17C.

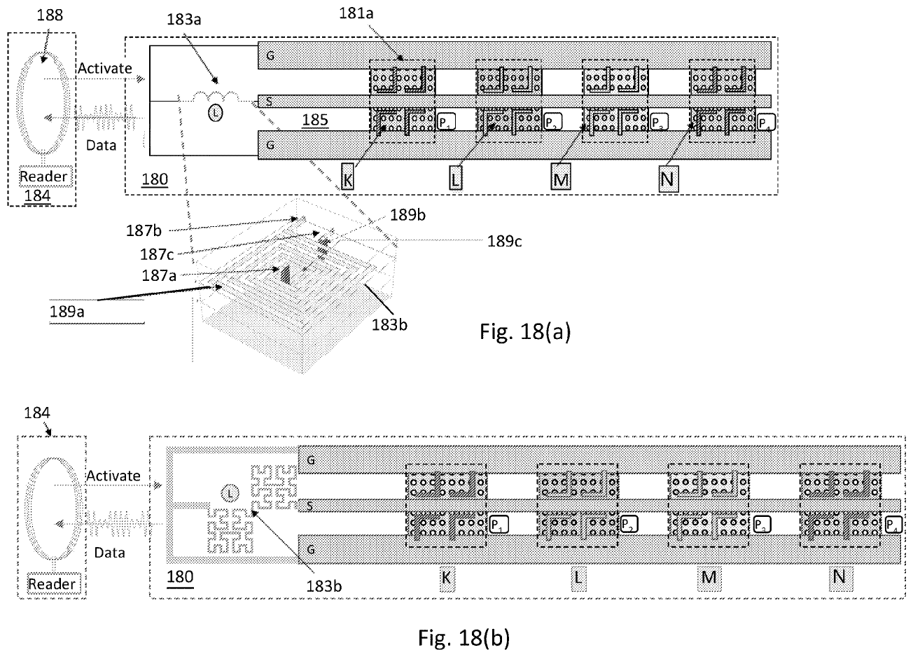

FIGS. 18A-B address different strategies for forming the inductor while maintaining a substantially planar geometry for the sensor. As discussed above, an inductor is used for coupling between the sensing component and a transmitter-receiver. This schematic illustration is reproduced in FIG. 18A. In FIG. 18A, transmitter-receiver 184, with its coupling element 188 is coupled to sensing device 180 through inductor 183a. Inductor 183a transmits a signal through coplanar waveguide 185, to sensing elements, of which one is identified as 181a. Inductor 183a is generally portrayed as a winding coil. The coil is either planar or of another configuration, but, as is well known, when winding a coil, it forms a spiral geometry in which one connection remains inside the coil and windings start at input 187a and go around until ending at contact 187b. However, to actually connect the coil to the circuit, a conductor must connect the internal contact to the outside of the coil conductor. For standard coils, this is generally not a problem, as a wire can be extended from where the internal coils start to the exterior of the coil windings area. For a planar coil, this is more challenging. This is because the coils are made of layers separated by an isolation material. For example, in FIG. 18A, coil 183b is composed of two metal layers. The coil starts at the connection 187b and winds, when this structure is fabricated on metal layer 189a, until it arrives to the internal end of coil 187a. In order to connect the inside end 187a to the outside line 187c, which is fabricated on metal layer 189a, something must be done. The solution is to fabricate a via or interconnect 189b that connects the metal in metal layer 189a with another metal layer 189c. In this form, the connection from coil end 187b goes through the interconnect to metal layer 189c which crosses (without shorting) all the coils' windings and then goes through a second interconnect again to metal layer 189a, ending in contact 187c. As can be understood, this requires a relatively complex fabrication procedure and the making of two metal layers, fabrication of vias, and an isolation layer that separates both metal layers, thereby preventing any short circuit. This complexity increases the cost of the device.

FIG. 18B illustrates a second embodiment of the sensing layer, with a different configuration of an inductor. In this embodiment, the inductor is a fractal inductor 183b. Transmitter-receiver 184 couples to sensor device 180, as previously described. Fractal inductor 183b uses a single metal layer, and does not require any vias or interconnects, or any changes of levels. The term “fractal inductor” refers to any structure that can be identified as fractal, or quasi-fractal, as defined by the fractal mathematical term, or any repeating or interwinding lines not crossing themselves in any point, that if fabricated from a conductive material can be considered to have inductance. Thus, a fractal inductor is a planar structure having inductance, serving as an inductor that can be drawn using only a single layer of metal without requiring crossing of itself or the use of interconnects or multiple layers of metal. The structure may be formed of a single segment or even of several segments spaced some distance from each other. Other possibilities to use and fabricate non-interwinding geometries for coupling elements are shown in additional examples such as in reference to FIG. 22B, etc.

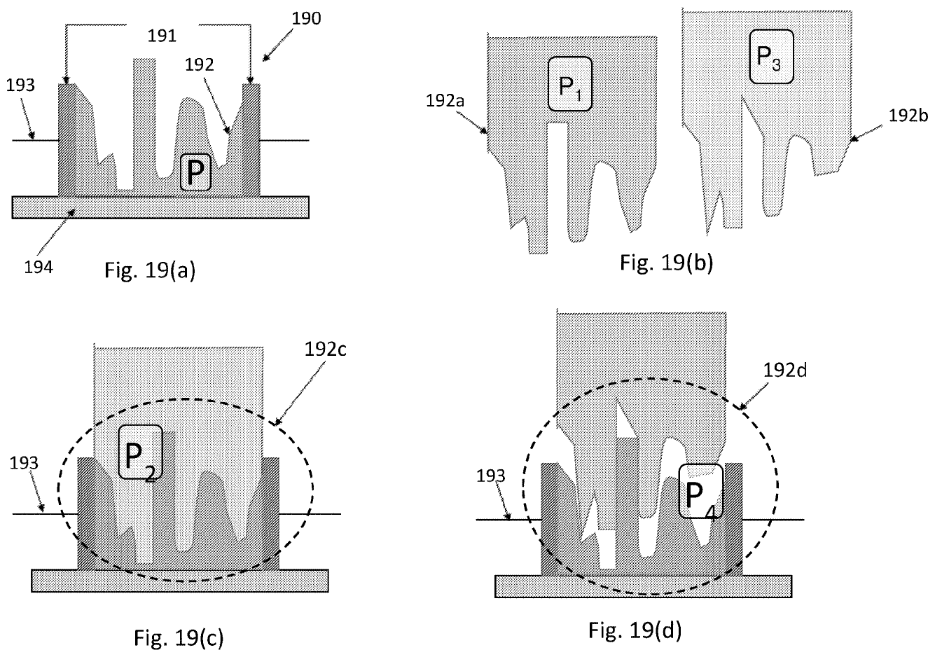

FIGS. 19A-21 illustrate manners in which the principles of the above-disclosure may be applied to general “materials” other than DNA-like strands. As discussed above, because the mechanism of testing relies on physical properties of the analyte material, the mechanism may be implemented for a wide range of materials. The following examples address the ways to make each sensor component to react to a specific sample to be tested, i.e. a “material,” in the broad concept of this word, when the “material” comes into sensing contact with the sensor zone, so as to change the cumulative material properties in a way that it shows a measurable difference. Examples will be given of selection of mechanical components, different chemical/biological/other solutions, chemicals etc., detection of radiation, so as to mention some of the possibilities beyond the example previously shown for detection of DNA like molecules.

Referring to FIGS. 19A-19D, the capacitance component of a device 190 is shown. As shown in FIG. 19A, the capacitor electrode plates 191 are connected to the rest of the circuit (not shown) by conducting lines 193 (schematic). The device may be held on a substrate 194. As indicated previously, between the electrodes a “material” 192 exists with properties P that affect the performance of the capacitor and therefore the natural frequency of the sensor. The material may fill all the gap/s between the electrodes and may have a geometrical configuration. As seen in FIG. 19B, there may be other sets of realized “materials” 192a and 192b etc. whose properties P1 and P2 etc. are the combination of their mechanical geometrical structure and the substances that they are composed of. What is important is that the overall configuration and substances are regarded as having certain properties, as indicated. If, as shown in FIG. 19C, material 192a with properties P1 is brought into sensing contact of the sensing element 190, and its geometrical configuration matches the one of the “materials” 192 already present between the capacitor plates, then both materials will combine forming a new “material” 192c with properties P2. It is clearly understood that this new “material” forms only if the geometries of both materials match. In this case, it is clear that it is the combination of the geometries and substance of both materials together that may provide the specific properties P2. Only when this combination is achieved, the specific overall properties attained will allow for the expected resonant frequency understood as a positive sensing or detection of “material” 192a. However, as is shown in FIG. 19D, “material” 192b which does not have a matching geometrical structural configuration or is composed from a different substance than the expected, its approach to the sensing zone will not complete the expected engagement either due to the mechanical inability to combine or due to the substance from which it is composed. The outcome will be that a different “material” 192d will be created with overall property P4. In this case the resonant frequency thus achieved will be different from the expected, thus either being disregarded from being a positive detection of the sensor or being indicated as a false or negative detection.

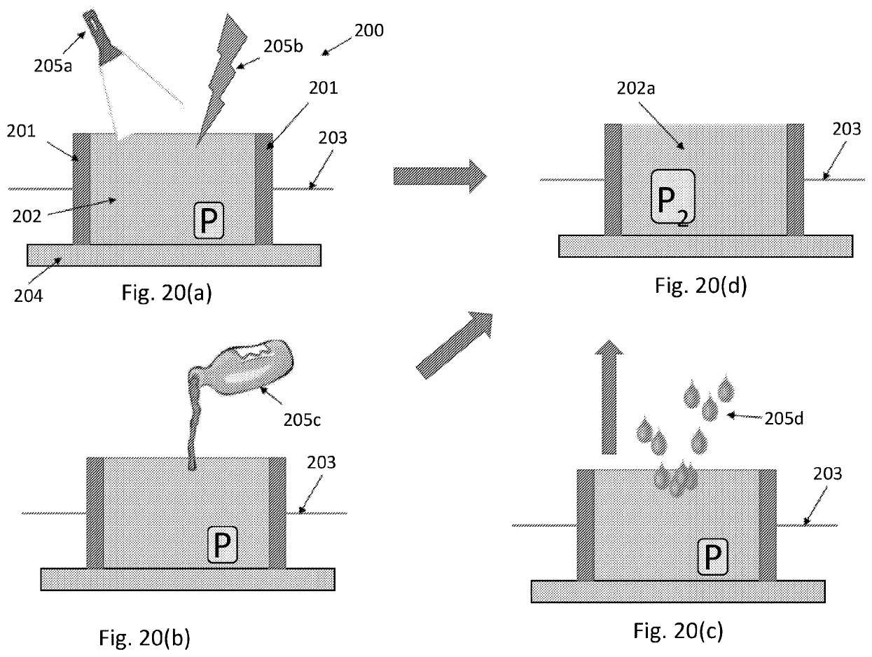

Other examples of “materials” as used in the broadest scope of the term are illustrated in respect to FIGS. 20A-20D. With reference to FIG. 20A, the capacitor element of the sensor of the device 200 is shown. The capacitor plates 201 are connected to the rest of the circuit (not shown) by conductors 203, and are optionally placed on a substrate 204. Between the plates, a material 202 with properties P is placed as to have a resonant frequency f. In FIG. 20A, the material 202 is such that it is sensitive to radiation. It is either sensitive to EM/light radiation 205a or to a radioactive radiation 205b, such as α, β, γ, or other kind of radiations. For example, a substance that can react to radiation can be a kind of photographic plate material or a photographic film etc. that reacts to the light and changes its properties, by for example, precipitating silver grains, which change the properties of the material. It is obviously possible to choose the substance and configuration of such materials as to respond to a specific “color” or type of radiation. After the radiation has been collected by the material, it effectively turns into a new material 202a with properties P2, as indicated in FIG. 20D, changing the material properties, and changing the capacitance and therefore the resonant frequency of the sensor, thus indicating a detection of the radiation being investigated. Notably, the frequency change in some cases is affected by the amount of change of one material into the other material. Thus, the frequency change may be a measure of the amount of radiation collected by the sensor, in addition to the simple positive detection.

Another embodiment of a “material” is explained in relation to FIG. 20B. In this case, material 202 with properties P can chemically react or bind or connect specifically to certain chemical substances. If such a chemical substance 205c is made to make contact with the material 202, a chemical or physical change will occur, and the outcome of this interaction will be a new material 202a with properties P2, as exemplified in FIG. 20D. This new material will change the frequency response of the sensor, thus detecting the presence of the intended substance. In case that substance 205c is not the intended substance to be detected, the chemical or physical change will not occur and thus the respective frequency will not change, and there will not be a detection signal. The reaction as described can be exemplified by way of the Benedict's Test, which is used to detect the presence of reducing sugars. In the test, a solution composed of copper sulfate, sodium carbonate and sodium citrate is seen as being a “material” 202, which has obviously some properties P. When the material to be tested 205c is glucose, which is an aldose whose open-chain forms an aldehyde group, and is placed together with material 202, a reaction occurs in which copper oxide precipitates and a carboxylic acid is formed. Therefore, new material 202a with properties P2 has been formed, which will induce a different frequency response of the filter. Other materials lacking the presence of the glucose will not trigger the reaction, thus not sending a detection signal.

Still, another variation of a “material” is explained in respect to the example depicted in FIG. 20C. In this example, material 202 is a hygroscopic material such as: aluminum hydroxide, graphene oxide, indium oxide, TiO₂ film, polyimide nanofibers, and polyethylene glycol etc. In case that moisture 205d exists, it adsorbs and is added to the material 202 and together effectively becomes a new “material” 202a with properties P2 per FIG. 20D, thus again changing the characteristics of the capacitor and with it the resonant frequency.

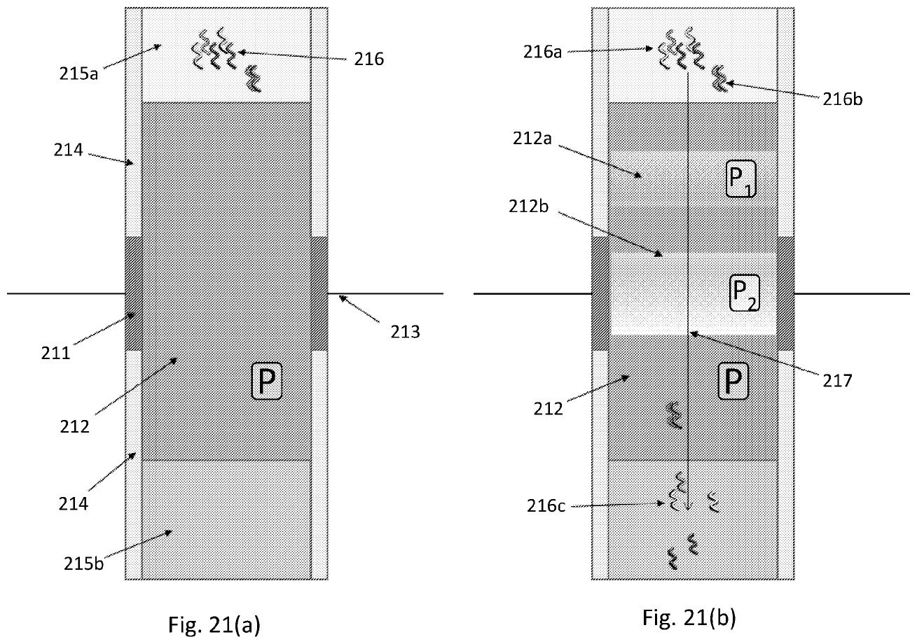

FIG. 21A illustrates usage of another type of material as a sensor which enables a continuous type of sensing. In FIG. 21A, a device without a substrate is shown. In this device, the capacitor plates 211 are connected to the other components (not shown) through wires 213. The plates are part of tube/conduit like structure that may be opened from both sides 214. At the capacitor plates zone and if needed also above and/or below it, a material 212 with properties P exists, which can be a gel agarose for example. DNA fragments (or any other suitable material for the methods described herein) are placed “above” material 212, in a reservoir with a solution 215a. These DNA fragments may be the consequence of a PCR procedure. At the bottom of the tube, another solution 215b is placed. If an electrical field is set between the upper and the lower sections of the tube (not shown), a motive force will drive the DNA fragments through, as it is well known in the art as electrophoresis. As it is shown in respect to FIG. 21B, the fragments 216, which are an aggregate of different types and sizes, have different dynamic properties and as such can be subdivided into groups per their dynamic capabilities such as 216a, 216b, 216c, etc. When motion starts, the DNA fragments will undergo a trajectory 217 from one side to the other, and become spaced and separated by their dynamic values. Those short DNA fragments will move fastest and traverse through the tube first, while the longer ones will follow in correspondence. While transiting through material 212, each species 216a, 216b, 216c, etc. will change locally the material 212 with property P into a temporary material 212a with properties P₁ and material 212b with properties P₂ and so on. As each new material passes along the location of the capacitor electrodes, eventually they will change the capacitance and therefore the response frequency of the resonator of the sensor. In this way, it will be possible to conclude, from each change from the original frequency due to material 212 to another frequency, the timing of arrival of each species. From this, it is possible to deduce which DNA fragment is involved and by this to map the DNA fragments in the sample. Furthermore, if the properties P₁ and P₂ are different for each group, then not only a change between two frequencies will occur, but many frequencies may occur at different times, with each frequency depending on the DNA fragment passing; thus, identifying the fragment by time and by frequency. As mentioned previously in regard to other embodiments, the concentration of the passing material wave might be a factor in the combined material property P₁, P₂ etc. Therefore, a quantitative indication based on the frequency of the response may be provided as well. Eventually, after transiting through the sensing zone, the species being tested will end up into a second reservoir 215b to be disposed there, as shown for fragments 216c.

In sum, while the preferred embodiments were given in greatest detail for DNA like fragments, other materials can be detected almost without any changes to the teachings besides the names of the materials. The resonators and the capacitors etc. may be identical whether DNA, or proteins, or a sugar solution, etc., is placed between the capacitor plates. It should be clear to those skilled in the art that the teachings here can be implemented for other materials, as long as they are placed at the sensors' capacitor or optical coupler gaps, according to the teachings of this invention.

What is claimed is:

1. A system for detecting DNA within a sample, comprising:
a plurality of transmitter-receivers configured to at least one of transmit and receive electromagnetic signals at a plurality of frequencies; and
a sensor device having a plurality of sensor elements coupled to the plurality of transmitter-receivers, such that electromagnetic signals are transmitted by a transmitter-receiver, pass through the sensor elements, and return to either the same or a different transmitter-receiver;
wherein each sensor element comprises a plurality of single-stranded DNA fragments, and each single-stranded DNA fragment of a sensor element is configured to couple to a specific single-stranded DNA fragment from the sample to form a double-stranded DNA fragment;
wherein, coupling of a single-stranded DNA fragment from the sample to a corresponding single-stranded DNA fragment from the sensor element causes a physical change in a range of signals that return to the same or the different transmitter-receiver.

2. The system of claim 1, wherein each sensor element comprises a resonance circuit including a capacitor and an inductor, said capacitor comprising a plurality of electrode plates, wherein the single-stranded DNA fragment of the sensor are adhered to the sensor element at a gap between the plates of the capacitor.

3. The system of claim 2, wherein each of the plurality of transmitter-receivers comprises a power source, a signal processor for converting power from the power source into electromagnetic signals, an oscillator for generating electromagnetic signals of a plurality of different frequencies, and a signal detector for measuring signals received by the transmitter-receiver.

4. The system of claim 3, wherein each of the plurality of transmitter-receivers further comprises a first inductive element, and the sensor device further comprises a second inductive element connected to the plurality of sensor elements, wherein the first inductive element and second inductive element are inductively coupled to each other, and wherein electromagnetic signals generated by a respective transmitter-receiver pass to the plurality of sensors of the sensing device via the coupled inductive elements, and wherein electromagnetic signals pass from the plurality of sensor elements of the sensor device to the signal detector of a respective transmitter-receiver via the coupled inductive elements.

5. The system of claim 4, wherein the second inductive element is a fractal inductor.

6. The system of claim 4, wherein the second inductive element is wired to a coplanar waveguide comprised of signal conductors and ground conductors, wherein each sensor element is coupled to at least one of the signal and ground conductors.

7. The system of claim 6, wherein, when a frequency of a signal transferred to a sensor device by the coplanar waveguide matches a frequency of the resonance circuit of a sensor element, the sensor element enters into resonance, and the resonance circuit acts as a band stop filter or band pass filter, thereby blocking transmission of the signal between the sensor device and the at least one transmitter-receiver, thereby causing a dip in signals of said frequency measured by the signal detector of said transmitter-receiver.

8. The system of claim 6, wherein, for each of the plurality of sensor elements, when the single-stranded DNA fragment from the sample is not coupled to the single-stranded DNA fragment from the sensor and therefore not forming a double-stranded DNA fragment, the band stop filter or band pass filter prevents transmission of a first resonant frequency to the transmitter-receiver, and when the single-stranded DNA fragment from the sample is coupled to the single-stranded DNA fragment from the sensor, the band stop filter or band pass filter prevents transmission of a second resonant frequency that is different from the first resonant frequency to the transmitter-receiver.

9. The system of claim 8, wherein each of the plurality of sensor elements has a different resonant frequency relative to the others of the plurality of sensor elements when a respective double-stranded DNA fragment is coupled therein, and wherein, when a transmitter-receiver transmits a range of signals having a plurality of frequencies, including frequencies in which one or more of the sensor elements has a resonant frequency, a signal detector detects each of the frequencies within the range at which a dip in power is measured.

10. The system of claim 2, wherein the single-stranded DNA of each sensor element is attached to beads which are placed between the plates of the capacitor.

11. The system of claim 10, wherein the beads are made of a conductive material.

12. The system of claim 11, wherein the beads are formed of a layer of conductive material that is coated by an isolating layer.

13. The system of claim 12, further comprising a filler material within the layer of conductive material.

14. The system of claim 10, wherein the beads are stacked within the gap to thereby create a packed structure of beads.

15. The system of claim 10, wherein, within each gap, a plurality of different types of single-stranded DNA fragments are attached to the beads in different ratios, thereby enabling determination of presence of more than one type of single-stranded DNA fragment from the sample, based on a magnitude of a change in capacitance.

16. The system of claim 2, wherein the electrode plates are arranged in one or more of the following configurations: parallel plate capacitor; interlacing comb of finger plates; or at least one electrode is a tip with a small apex.

17. The system of claim 16, wherein the capacitor is a parallel-plate capacitor and the single-stranded DNA is adhered between the plates of the parallel-plate capacitor.

18. The system of claim 1, wherein the plurality of transmitter-receivers are configured to transmit and receive optical signals; the sensor device comprises an optical transmission line for transmitting and receiving optical signals, each sensor element comprises an optical coupler including a gap between two adjacent waveguides, wherein the optical coupler permits transfer of light of a particular coupling frequency across the gap, and wherein the single-stranded DNA of each sensor element is adhered within the gap.

19. The system of claim 18, wherein, when a frequency of a signal being transferred through the optical transmission line matches the coupling frequency of the optical coupler of a given sensor element, the signal at that frequency is transferred between the waveguides of said sensor element, thereby preventing further transmission of the signal along the transmission line, causing said frequency to not be received by a respective transmitter-receiver.

20. The system of claim 19, wherein, for each of the plurality of sensors, when the single-stranded DNA fragment from the sample is not coupled to the single-stranded DNA fragment from the sensor, the optical coupler prevents transmission of a first coupling frequency to the transmitter-receiver, and when the single-stranded DNA fragment from the sample is coupled to the single-stranded DNA fragment from the sensor, the optical coupler prevents transmission of a second coupling frequency that is different from the first coupling frequency to the transmitter-receiver.

21. The system of claim 20, wherein each of the plurality of sensor elements has a different coupling frequency for the optical coupler relative to the others of the plurality of sensor elements when a respective double-stranded DNA fragment is configured in the gap, and wherein, when the transmitter-receiver transmits light in a plurality of frequencies, including coupling frequencies of each of the optical couplers, the signal detector detects each of the frequencies at which transmission is prevented.

22. The system of claim 18, wherein advancing of a light signal through the optical transmission line induces a secondary light emission due to phosphorescence or fluorescence induced on the single-stranded or double-stranded DNA of each sensor element, wherein the secondary light emission is detectable by a signal detector of a transmitter-receiver.

23. The system of claim 18, wherein the plurality of transmitter-receivers are configured to emit and receive light of multiple frequencies simultaneously.

24. The system of claim 1, wherein introduction of material to the sensor elements that is not a single-stranded DNA sample capable of coupling to the single-stranded DNA of the sensor element causes no change in a range of signals that return to the same or different transmitter-receiver, or a different change in the range of signals that return to the same or different transmitter-receiver compared to introduction of the specific single-stranded DNA fragment from the sample.

25. A method for detecting DNA within a sample, comprising:
transmitting electromagnetic signals from a plurality of transmitter-receivers to a sensor device containing a plurality of sensor elements, wherein the sensor elements are coupled to the plurality of transmitter-receivers such that electromagnetic signals are transmitted by a transmitter-receiver, pass through the sensor elements, and return to either the same or a different transmitter-receiver; wherein each sensor element comprises a single-stranded DNA fragment, and said single-stranded DNA fragment of the sensor element is configured to couple to a specific single-stranded DNA fragment from the sample to form a double-stranded DNA fragment, wherein, coupling of a single-stranded DNA fragment from the sample to a corresponding single-stranded DNA fragment from the sensor element causes a physical change in a range of signals that return to the same or a different transmitter-receiver;
detecting the electromagnetic signals received by the plurality of transmitter-receivers; and
based on whether a particular electromagnetic signal that was sent by a transmitter-receiver is subsequently received by the same or a different transmitter-receiver, determining whether a particular single-stranded DNA fragment from the sample is coupled to a corresponding single-stranded DNA fragment from the sensor element.

26. The method of claim 25, wherein:
each sensor element comprises a resonance circuit including a capacitor and an inductor, and wherein the single-stranded DNA of the sensor element is adhered to the sensor element at a gap between the plates of the capacitor;
each transmitter-receiver comprises a power source, a signal processor for converting power from the power source into electromagnetic signals, an oscillator for generating electromagnetic signals of a plurality of different frequencies, and a signal detector for measuring power received by the transmitter-receiver;
a first inductive element is in each respective transmitter-receiver and at least one second inductive element is within the sensor device and connected to the plurality of sensors, wherein each first inductive element is inductively coupled to a second inductive element;
and wherein the method further comprises: generating electromagnetic signals in at least one of the transmitter-receivers; passing the electromagnetic signals from the transmitter-receiver to the sensor device via an inductive coupling between the first inductive element of said transmitter-receiver and a second inductive element; and passing the electromagnetic signals, via an inductive coupling, from the plurality of sensor elements to the signal detector of the same or a different transmitter-receiver.

27. The method of claim 26, wherein, when a frequency of a signal transferred to the sensor device by the first and second inductive elements matches a frequency of the resonance circuit of a sensor element, the sensor element enters into resonance, and the resonance circuit acts as a band stop filter or band pass filter, thereby blocking transmission of the signal to the signal detector, causing a dip in power measured by the signal detector.

28. The method of claim 27, further comprising, for each of the plurality of sensor elements, when the single-stranded DNA fragment from the sample is not coupled to the single-stranded DNA fragment from the sensor, preventing transmission of a first resonant frequency to the transmitter-receiver, and when the single-stranded DNA fragment from the sample is coupled to the single-stranded DNA fragment from the sensor, preventing transmission of a second resonant frequency that is different from the first resonant frequency to the transmitter-receiver.

29. The method of claim 28, wherein each of the plurality of sensor elements has a different resonant frequency relative to the others of the plurality of sensor elements when a respective double-stranded DNA fragment is configured between plates of the capacitor, and the method further comprises:
transmitting, with at least one of the plurality of transmitter-receivers, a range of signals of a plurality of frequencies, including frequencies in which one or more of the sensor elements has a resonant frequency; and
detecting, with the signal detector of at least one of the transmitter-receivers, each of the frequencies at which a dip in power is measured.

30. The method of claim 25, wherein:
the plurality of transmitter-receivers are configured to transmit and receive optical signals;
the sensor device comprises an optical transmission line for transmitting and receiving optical signals;
each sensor element comprises an optical coupler including a gap between two adjacent waveguides, wherein the optical coupler permits transfer of light of a particular coupling frequency across the gap, and wherein the single-stranded DNA of each sensor element is adhered within the gap;
and the method further comprises:
generating optical signals with at least one of the plurality of transmitter-receivers; transferring the optical signals from the at least one transmitter-receiver to the plurality of sensor elements via the optical transmission line; and transferring the optical signals from the optical transmission line to the signal detector of at the at least one transmitter-receiver or a different transmitter-receiver;
wherein, when a frequency of a signal being transmitted through the optical transmission line matches the coupling frequency of the optical coupler of a given sensor element, the signal at that frequency is transmitted between the waveguides of said sensor element, thereby preventing further transmission of the signal along the transmission line, causing said frequency to not be received by a respective transmitter-receiver.

31. The method of claim 30, further comprising, for each of the plurality of sensor elements, when the single-stranded DNA fragment from the sample is not coupled to the single-stranded DNA fragment from the sensor element, preventing transmission of a first coupling frequency to the signal detector, and when the single-stranded DNA fragment from the sample is coupled to the single-stranded DNA fragment from the sensor, preventing transmission of a second coupling frequency that is different from the first coupling frequency to the signal detector.

32. The method of claim 31, wherein each of the plurality of sensor elements has a different coupling frequency for the optical coupler relative to the others of the plurality of sensor elements when a respective double-stranded DNA fragment is configured in the gap, and further comprising: transmitting light with the at least one transmitter-receiver in a plurality of frequencies, including coupling frequencies of each of the optical couplers, and detecting, with the signal detector, each of the frequencies at which transmission is prevented.

33. The method of claim 30, further comprising: inducing a secondary light emission due to phosphorescence or fluorescence on the single-stranded or double-stranded DNA of each sensor element; and detecting the secondary light emission by a signal detector of a transmitter-receiver.

34. The method of claim 30, further comprising emitting and receiving light of multiple frequencies simultaneously.

35. A system for detecting a sample material within an agglomerate of sample materials, comprising:
a sensing device comprising a plurality of detecting elements, each detecting element being specific for a particular material to be detected, and
one or more external transmitter-receivers;
wherein the sensing device is configured to output a detecting signal to the one or more external transmitter-receivers, signifying a positive detection of the material to be detected, when an introduced corresponding material matches the first material in at least one of geometrical configuration and chemical properties, and the sensing device is configured to not output the detecting signal signifying a positive detection to the one or more external transmitter-receivers when the introduced corresponding material does not match the first material in at least one of geometrical configuration and chemical properties.

36. The system of claim 35, wherein each detecting element comprises a capacitive element including actual or theoretical electrodes or plates; and a first material between the electrodes or plates, wherein the presence and amount of the first material measurably affects a capacitance of the capacitive element, and wherein introduction of a corresponding material to be detected between the electrodes or plates, and thereby generating of a second material comprising the first material and the material to be detected, measurably affects the capacitance of the capacitive element relative to the capacitance of the detecting element in a presence of only the first material, causing a difference in measured parameters of capacitance.

37. The system of claim 36, wherein the first material is a light or radiation sensitive substance that is sensitive to a particular range of radiation, and the material to be tested is radiation, wherein introduction of radiation of a particular spectrum changes properties of the first material, which thereby affects the measured parameters of capacitance.

38. The system of claim 36, wherein the first material is a chemical substance that is sensitive to reaction with a specific chemical substance to be tested, and the material to be tested is a chemical substance, wherein introduction of the chemical substance to be tested induces a chemical reaction in the first material, which thereby affects the measured parameters of capacitance.

39. The system of claim 36, wherein the first material is a hygroscopic substance that is reactive to presence of water drops or vapor, and the material to be tested is water drops or vapor, wherein introduction of the water drops or vapor induces a chemical reaction in the first material, which thereby affects the measured parameters of capacitance.

40. The system of claim 36, wherein the sensing device further comprises a channel between the electrodes or plate, the first material is positioned at a specific location within the channel, the channel comprises an inlet and an outlet, and a means for advancing materials from the inlet to the outlet, wherein, when corresponding material to be tested is drawn from the inlet to the outlet, a change in capacitance is induced when the material to be tested reaches and interacts with the first material.

41. The system of claim 40, wherein the first material is configured within a gel substance, the material to be tested is a DNA-like fragment, and the means for advancing materials is electrophoresis.
